(19) United States
(12) Patent Application Publication
(10) Pub. No.: US 2019/0201225 A1
(43) Pub. Date: Jul. 4, 2019

(54) STENT DELIVERY SYSTEMS WITH SHAPED EXPANSION BALLOONS

(71) Applicant: Elixir Medical Corporation, Milpitas, CA (US)

(72) Inventors: Motasim Sirhan, Los Altos, CA (US); Udayan G. Patel, San Jose, CA (US)

(73) Assignee: Elixir Medical Corporation, Milpitas, CA (US)

(21) Appl. No.: 16/293,406

(22) Filed: Mar. 5, 2019

Related U.S. Application Data
(63) Continuation of application No. PCT/US2017/049308, filed on Aug. 30, 2017.
(60) Provisional application No. 62/408,016, filed on Oct. 13, 2016.
(60) Provisional application No. 62/393,423, filed on Sep. 12, 2016.

(51) Int. Cl. A61F 2/958; A61L 29/06; A61L 29/14

(52) U.S. Cl. CPC A61F 2/958; A61M 25/1002; A61L 29/14; A61L 29/06

(57) ABSTRACT

A stent or other luminal prosthesis is delivered by a catheter having a contoured balloon. The contoured balloon may include a central dome region flanked by at least one adjacent flat or cylindrical region. The central domed region and adjacent flat or cylindrical regions are joined at shallow angles to provide for an incrementally larger expansion of the center region of the stent while minimizing shear forces during expansion.

### CROSS-REFERENCE TO RELATED APPLICATIONS

This application is a continuation of PCT Application No. PCT/US2017/049308 (Attorney Docket No. 32016-713.601), filed Aug. 30, 2017, which claims the benefit of U.S. Provisional No. 62/408,016 (Attorney Docket No. 32016-713.102), filed Oct. 13, 2016, and U.S. Provisional No. 62/393,423 (Attorney Docket No. 32016-713.101), filed Sep. 12, 2016, the entire content of which are incorporated herein by reference.

### BACKGROUND OF THE INVENTION

### 1. Field of the Invention

The present invention relates generally to medical devices and treatment methods. More particularly, the present invention relates to scaffolds such as stents and grafts and the delivery of such scaffolds to the vasculature using a delivery catheter with a balloon having desirable characteristics.

Balloon angioplasty is introduced to open vessels, particularly blood vessels which have narrowed as a result of plaque progression or a heart attack. In successful cases, the blood vessel remains open and may exhibit positive remodeling over time and/or vasodilation ability mimicking to a degree the natural vessel ability. In other cases, however, the blood vessesl will re-occlude within days or months due to various causes such as recoil of the vessel, thrombus formation, or the type of plaque morphology or progression.

Metallic scaffolds were developed to provide a structure, often referred to as a stent, with sufficient strength to address vessel recoil and hold the vessel open over time. Stents have been formed as coils, braids, and tubular bodies. Balloon expandable stents formed from patterned metallic tubes are now most commonly used as they display desirable structural characteristics such as limited recoil, high strength (crush resistance), and limited axial shortening upon expansion, when compared to coiled or braided stents.

Despite their success and widespread adoption, metallic stents suffer from certain shortcomings, such as preventing the lumen or vessel from further expanding which in turn inhibits positive remodeling and/or vasodilation of the treated vessel which is important to healing of the vessel. This phenomenon is commonly referred to as “jailing” or “caging” the vessel.

To address this shortcoming, biodegradable stents or scaffolds made from metallic or polymeric materials were developed. By allowing the stent to degrade or resorb, the jailing effect will diminish and finally disappear over time. Present biodegradable stents, however, have their own shortcomings, including stent fractures, excessive recoil, and/or insufficient strength to accommodate various lesion types to name a few.

Stents, including polymeric and metallic biodegradable stents, are often deployed with a balloon catheter having a constant balloon diameter to deploy the stent with a nominal (or labeled) diameter. Catheters for deployment of stents typically have semi-compliant or non-compliant, cylindrical balloons formed from a generally non-distensible or non-compliant material, such as Nylon, and poly(ethylene terephthalate) (PET). The advantage of these balloons is that they achieve a substantially uniform inflated profile at a particular selected pressure.

When treating some calcified lesions with a constant diameter semi-compliant or noncompliant balloon and constant diameter stent, the rigid calcification of the lesion can lead to non-uniform expansion of the stent. In some cases, the ends of the balloon and/or stent will be fully deployed while the center of the stent and/or balloon can be less than fully deployed resulting in the deployed stent having an hourglass or “dog bone” shape. This hourglass shape causes flow restriction and can result in thrombus, reocclusion, and/or restenosis.

What is needed is a stent delivery system or combination of stent delivery system and stent that addresses at least some of these issues.

### 2. Listing of Background Art

Relevant background patents and applications include:

U.S. Pat. Nos. 5,338,298; 5,470,313; 6,221,043; 6,432,080; 6,872,215; 7,037,318; 7,736,362; 8,251,942; 8,715,228; 8,945,160; 8,333,795; 8,309,007; 7,122,019; 6,383,212; 6,352,551; 4,777,951; 7,186,237; 7,862,495; 5,645,560; 7,843,116; 7,467,243; 5,609,605; 5,749,851; 8,333,795; 4,777,951; 6,383,212; 7,122,019; 8,309,007; 8,956,399; 8,747,453; 8,524,132; 7,731,742; 5,922,019; US2004/0267350; and US2005/0049671.

### SUMMARY OF THE INVENTION

The present invention provides stent delivery systems including stents and stent delivery catheters. The stent delivery systems of the present invention are useful for delivering stents, grafts, and other luminal prostheses to blood vessels and other body lumens. The stent delivery systems of the present invention are particularly useful for delivery polymeric vascular stents, biodegradable polymeric stents, and stents with separation regions, such as those described in commonly owned PCT Patent Application PCT/US2016/026821 (Attorney Docket No. 32016-712.604 (590)), and commonly owned U.S. patent application Ser. No. 12/016,085 (Attorney Docket No. 32016-712.202 (520)); U.S. patent application Ser. No. 14/604,621 (Attorney Docket No. 32016-712.202 (530)); U.S. patent application Ser. No. 14/800,536 (Attorney Docket No. 32016-712.203 (580)); and U.S. patent application Ser. No. 15/605,601 (Attorney Docket No. 32016-714.301), the full disclosures of which are incorporated herein by reference.

The stent delivery catheters of the present invention include a catheter body having a stent delivery balloon at or near a distal end thereof. The stent delivery balloon has a longitudinal profile with a central region and at least one, or a pair of flanking regions. The central region will have a convex or dome-shaped surface or region when inflated which, when used to deliver a stent or other prosthesis, will promote non uniform shape, convex shaped, dome-shaped, non-hour-glass, non-dog-bone, and/or full expansion, of the stent or other prosthesis within air, water, water at 37° C., and/or the body lumen being treated. In particular, the combination of a convex central region with flanking regions which are flat, or relatively less convex than the central region, or concave, will at least partially overcome the tendency of polymeric and/or other stents and prostheses to form an “hourglass” or “dog bone” expansion configuration in vessels such as calcified or fibrotic blood vessels and other body lumens.

While the use of “stepped” balloons having a raised region or “plateau” formed in a region of the balloon for stent delivery is known, such stepped regions present an abrupt transition between the raised region and the adjacent regions of the balloon. Such abrupt transitions will subject the stent being expanded by the balloon to significant shear forces and/or stresses as the balloon is inflated to expand the stent which might cause stent fractures and/or edge dissections. While such shear forces and/or stresses may be acceptable for some metallic stent structures, they are problematic for many if not all polymeric stents, and in particular for biodegradable (including bio-corrodible and bio-resorbable) stents such as polymeric stents.

The present invention provides a convex central region, defined and illustrated below, which is flanked by at least one and usually two less convex (usually flat or substantially flat and more usually cylindrical) flanking region(s). The convex central region and each adjacent flanking region are joined by at least one transition region which connects the convex central region and the flanking region at a transition an angle α (defined below) ranging from 125° to 179°, preferably 150° to 179°, more preferably from 170° to 179°, often from 175° to 178.5°, and usually from 176° to 178°.

Usually, for coronary balloons having a nominal diameter (or labeled diameter) in the range from 2.5 mm (millimeters) to 4.0 mm, the maximum diameter of the convex central region will be incrementally larger by from 0.11 mm to 1 mm, typically from 0.13 mm to 0.5 mm, more typically from 0.15 mm to 0.35 mm. As a percentage, the maximum diameter of the inflated convex central region will usually be from 3% to 17% larger than the nominal inflated diameter of the balloon, usually being from 3% to 15% larger than the nominal inflated diameter of the balloon, and often being from 4% to 15% larger than the nominal diameter of the balloon.

Usually, for peripheral balloons having a nominal diameter (or labeled diameter) in the range from 4.5 mm (millimeters) to 20 mm, the maximum diameter of the convex central region will be incrementally larger by from 0.25 mm to 2 mm, typically from 0.5 mm to 1.5 mm, more typically from 0.75 mm to 1 mm. As a percentage, the maximum diameter of the inflated convex central region will usually be from 3% to 30% larger than the nominal inflated diameter of the balloon, usually being from 4% to 20% larger than the nominal inflated diameter of the balloon, and often being from 5% to 17% larger than the nominal diameter of the balloon.

Usually, for coronary balloon and/or stent lengths ranging from 10 mm to 50 mm, typically from 14 mm to 40 mm, and more typically from 18 mm to 38 mm, the length of the flanking region ranges from 0.1 mm to 10 mm, preferably from 0.5 mm to 6 mm, more preferably ranges from lmm to 4 mm.

The nominal diameter of the balloon will typically be the diameter of the adjacent flanking region or regions, typically taken at a location adjacent to the transition region and the central region of the balloon. Alternatively, the nominal diameter of the balloon may be the average diameter of one or both flanking regions taken partially or fully along their length(s), and/or the nominal diameter of the balloon will be approximately the diameter or average diameter of a distal flanking region, and/or the nominal diameter of the balloon will be approximately the diameter or average diameter of a proximal flanking region, and/or the nominal diameter may be a labeled diameter of the delivery system, and/or stent. The phrase “nominal diameter” usually refers to the diameter measured when the balloon is inflated to its expected or nominal inflation pressure.

This combination of (1) a transition region having a preselected transition angle, (2) a convex central region (preferably being convex across the length of the central region) having a maximum diameter (along the length of said convex region) which is a small percentage greater than the nominal diameter, and (3) at least one flanking region, has been found to provide a number of benefits including improved expansion of stents, reduced stent under-deployment (under-expansion), reduced stent under-deployment (under-expansion) over at least a portion of the convex central region, reduced dissection of the vessel, reduced edge dissection, reduction in the hourglass profile of the expanded stent or balloon, reduced damage or fracture to the stent by shear forces or other causes, increased ability to expand the stent without fracture, increased ability to expand the stent to a “rated burst pressure” of the delivery system without stent fracture, increased ability to expand the stent in the convex central region of the balloon without fracture, and/or an ability to achieve optimal deployment of the stent.

In a first aspect of the present invention, a stent delivery catheter comprises a catheter body having a proximal end, a distal end, and a longitudinal axis. An inflatable balloon is mounted on the catheter body near the distal end and has a central region, a proximal flanking region, and a distal flanking region. The central region is convex relative to the flanking regions (i.e. more convex that the flanking region(s) which are typically flat) when viewed in profile along the longitudinal axis and, each flanking region may be joined to the convex central region at a transition angle a which may be in a first set of range from 125° to 179°, often from 135° to 179°, often from 150° to 179.5°, preferably 160° to 179°, more preferably, 170° to 179°, and still more preferably from 170° to 178°, often from 175° to 178° when inflated. Alternatively, each flanking region may be joined to the convex central region at a transition angle α which may be in a second set of ranges from 125° to 179°, preferably ranging from 135° to 179°, more preferably ranging from 150° to 179°.

In particular embodiments or examples, the convex central region will have a spheroidal or ellipsoidal surface when inflated. By “spheroidal” or “ellipsoidal,” is meant that the surfaces on the inflatable balloon will be truncated or substantially truncated annular portions of a sphere or ellipse, respectively. Such annular truncations are illustrated in the Detailed Description hereinbelow. Usually, the spheroidal or ellipsoidal profile of the convex central region will be uniformly curved between the distal and proximal flanking regions. That is, the surfaces will follow a true spheroidal or ellipsoidal line along the entire length of the convex central region. In other instances, however, the spheroidal or ellipsoidal surface may have a greater or lesser curvature at or near its proximal and/or distal regions where it joins the flanking regions. In still other instances, the convex central region may be spheroidal or ellipsoidal over distal and/or proximal lengths thereof while being flatter or substantially flat over some length thereof.

In one example, a spheroidal balloon shape having convex central region and a transition angle to at least one of proximal and/or distal flanking regions, said angle ranging from 170° to 179°, preferably ranging from 175° to 178°. In another example, an ellipsoidal balloon shape having a convex central region and a transition angle to at least one of proximal and/or distal flanking regions, said angle ranging from 125° to 170°, preferably ranging from 135° to 170°, more preferably ranging from 150° to 170°.

In one example, the convex central region transition angle to the distal flanking region may be different from the convex central region transition angle to the proximal flanking region. In another example, the proximal and distal transition angles may substantially the same. In another example, the convex central region may contain a flat or substantially flat region along a length or segment of the convex central region. In another example, a flat or substantially flat segment or portion of the central convex region may be tapered such that one end of the flat or substantially flat segment or portion is larger than the other end. In another example, the diameter or mean diameter of the proximal flanking region may be substantially the same as the diameter or mean diameter of the distal flanking region. In another example, the proximal and distal diameters of the flanking regions may be different.

In one example, the inflatable balloon of any of the examples is a balloon dilatation catheter.

In a preferred example, the flanking region length from the nominal inflated pressure to RBP remains substantially the same length. In another example, the flanking region length from nominal pressure to RBP pressure decreases by 1-2 mm. In a third example, the flanking region length from nominal pressure to RBP pressure maintains at least a 0.5 mm to 4 mm flanking region length. In a fourth example, at least a portion of the flanking region is maintained when the balloon is inflated at pressure ranging from nominal pressure to RBP pressure.

In a specific example, the inflatable balloon having a convex central region larger than at least one adjacent proximal and/or distal flanking region diameter, wherein the transition angle from the central region to the distal region, the transition angle from the central region to the proximal region, and the length of the distal and proximal regions (adjacent to the central region), control the rate or magnitude of diameter of the proximal region compared to the central region and/or the distal region in the inflated balloon condition, or at pressures ranging from nominal to the rated balloon pressure (RBP) or burst pressure. For example, it is desired to have a proximal region, adjacent to the central convex region of an inflatable balloon, to have larger diameters than the distal region in the inflated condition, or at certain pressures such as nominal, RBP, or a range from nominal to RBP. The proximal transition angle (central region to adjacent proximal region) can for example be in the range from 150° to 170° and the proximal region length can be 2 mm. The distal transition angle (central region to adjacent distal region) can be in the range from 170° to 179° and the distal region length can be 3 mm. The proximal region diameter at an inflated pressure, or at nominal pressure, or at RBP pressure, or at pressures ranging from nominal to RBP, can be larger in the proximal region than the distal region for desired length as the pressure increases. For example, at nominal pressure, the proximal region length is 2 mm and the diameter for example is 3.0 mm, and the distal region length is 3 mm and the diameter is also 3 mm. The measurements at RBP can be as follows: the proximal length can be 1 mm and the diameter for example be 3.35 mm (at least in one region of the proximal region adjacent to the central region) while the distal region length can remain substantially 3 mm in length and have a diameter of 3.3 mm. This allows a user to control the proximal region diameter at certain pressures or as the pressure increases from nominal to RBP. It also allows control of the proximal region length and diameter relative to the central region.

In some examples the angles, mean angles, diameters, mean diameters, lengths, widths, thicknesses, and other measurements, are measure in the inflated balloon condition, nominal inflated (or labeled) diameter, at about RBP, and/or at any pressure in between.

In one example, the convex central region of the balloon is at least in part formed from a plurality of discrete steps substantially forming a convex shape across the length of the central region, typically at least three discrete steps, often at least five discrete steps, and sometimes seven or more discrete steps, where the outer most step or steps will form transition regions, as defined elsewhere herein, with the adjacent flanking regions transition regions. In another example, a flat region or substantially flat region, or a second convex or dome region having a different curvature, can be formed along the length of the convex central region, preferably about the center of the central convex region. The central convex region can have a center or region of maximum diameter which is positioned proximally or distally of the center point of the balloon and/or the center region, or can be positioned substantially in the middle of the balloon length.

In other particular examples and/or embodiments, the surface of the convex central region may be smooth when inflated. In still other particular examples and embodiments, the surface of the convex central region may be textured when inflated, and a variety of particular texturing features are described in detail hereinbelow, and include corrugations, bumps, saw tooth elements, ribs, and the like.

Typically, the flanking regions will be cylindrical, but in other examples and embodiments may be tapered, for example either increasing or decreasing in diameter in a direction away from the central region of the balloon. In still other particular examples and embodiments, the flanking region may have a smooth surface or may have a textured surface similar to or different from that of the convex central region. In some instance, the flanking regions themselves may have a small curve or convexity, but the curvature will usually be much less than that of the central convex region. In particular, when tapered, the flanking regions will typically have a taper angle β relative to the axial direction which is much less than the angle γ relative to the axial direction at which the convex central region joined the flanking regions. In all cases, the transition angle α will be maintained within the ranges set forth above. These angles are defined and discussed with reference to FIG. 2 below.

The inflatable balloons of the present invention may be formed from materials which are conventional for the fabrication of stent delivery catheter balloons. For example, the inflatable materials may be formed from one or more non-compliant polymers, such as polyethyleneterphthalate, polyamideimide copolymer, polyetherimide, polyetherketone, polyetheretherketone, polybutyleneterphthalate, polycarbonate, polyacetate, polyphthalamide, polycrylonitrile, polyarylene, polybutadiene, polyether, polyetherketones, polyimide, polyphenylenesulfide, polyphosphazenes, polyphosphonates, polysulfone, polycarbonate/polysulfone alloy, polysulfides, polsulfide, polythiophene, polyacetylene polycarbonates, polyphenylene ether, polyetherketones, polyimide, polyphenylene, Polycarbonate/polybutylene terephthalate alloy, ABS/PC blend, carbon reinforced composites, aramid fiber reinforced composites, poly [(R)-3-hydroxybutyrate-co-8%-(R)-3-hydroxyvalerate](P (3HB-co-8%-3HV)fibers composites, liquidcrystal fibers composites, blends and/or combinations thereof.

Alternatively, the inflatable balloons may be formed at least in part from one or more semi-compliant polymers, such as polyamide (nylon 12, nylon 11, nylon 6-12, nylon 6-11, nylon 6-6, nylon 6,), polyetheramide block copolymers, nylon blends, nylon copolymers, polyurethane, polyesterpolyurethane, poycarbonatepolyurethane, polyetherpolyurethane, polyolefinpolyamide, polyacrylonitrile, polytrimethyleneterephthalate, polyacrylonitrilebutadienestyrene, polyphenylsufone, polyphthalamide, polyaryletherketone, polyethersulfone, polybutyleneadipate, polyacetate, polyacrylate, ABS/Nylon blends, polycrylonitrile, polyanhydride, polyarylene, combinations, and/or blends. In other examples blends and/or combinations of one or more noncompliant polymers, one or more semi-compliant materials, and one or more compliant materials can comprise the balloon material.

Usually, the convex central region and the distal and proximal flanking regions will have the same, similar, or substantially similar compliance, although in alternative embodiments or examples they may have different compliances or be formed from materials having different compliances. Also typically, the inflatable balloons of the present invention may have a substantially uniform wall thickness but in other instances may have a non-uniform wall thickness. For example, the convex central region of the inflatable balloon may be thinned or thinner relative to other portions of the balloon in order to achieve the desired convex inflation geometry.

Alternatively, the inflatable balloon may include additional layers, restraints, limiting members, or other additive features which can control the inflated shape of the balloon including both the expanded convex geometry of the central region as well as the flat, tapered, or other geometries of the flanking regions. In cases where the balloon has a substantially uniform wall thickness, the geometry of the balloon will usually be achieved by molding the balloon into the desired geometry with the substantially uniform wall thickness. Where the wall thickness of the convex central region is thinned or thinner, such thinning may, for example, be achieved by heat shaping of the balloon after the balloon is initially molded or otherwise fabricated.

In a preferred example, prior to inflation, the balloons of the present invention will usually be folded into a generally cylindrical configuration having a substantially uniform diameter and circumference over substantially the entire length of the balloon. While the diameter and circumference may vary to a minor degree because of differences in wall thickness or other factors, these differences will be minor compared with the differences in geometry and dimensions among the various regions of the balloon when the balloon is inflated or fully inflated. Additionally, in particular examples and embodiments, the balloon will retain its desired geometry with the enlarged convex central region and smaller adjacent flanking regions at substantially all inflation pressures expected for its intended use, typically at pressures from nominal to the rated burst inflation pressure.

In certain examples and/or embodiments, the central convex region of the inflatable balloon will have a length which is greater than or equal to 40% of a length of the inflatable balloon, where the length of the inflatable balloon is typically measured between a distal end of the distal flanking region to a proximal end of the proximal flanking region, and/or the length of the inflatable balloon is the length which has a diameter equal to or larger than the labeled (or nominal) diameter of the stent/delivery system when the balloon is inflated to the labeled (or nominal) diameter pressure, and/or the length of the inflatable balloon which is the working length of the balloon. That is, the length of the balloon will not include the conical or other end regions of the balloon which taper down to the catheter body or shaft. In other examples and embodiments, the central convex region will have a length equal to or greater than 50% of the length of the inflatable balloon, and in still other examples and embodiments the central convex region will have a length equal to or greater than 60% of the length of the inflatable balloon. In other examples, the central convex region of the inflatable balloon will have a length ranging from 30% to 95% of the inflatable balloon length, preferably ranging from 40% to 85% of the inflatable balloon length, more preferably ranging from 50% to 80%.

In still further examples and embodiments of the present invention, the central convex region of the inflatable balloon is larger than the distal and/or proximal flanking regions when the balloon is inflated to pressures from 1 to 10 atm in air, water, water at 37° C., and/or under physiological conditions, often maintaining substantially the same or similar geometry when inflated to pressures from 1 to 40 atm in air, water, water at 37 ° C., and/or under physiological conditions, more often maintaining substantially the same or similar geometry when inflated to pressures from nominal (labeled) to rated burst pressure (atm) in air, water, water at 37° C., and/or under physiological conditions. Usually, the central convex region of the inflatable balloon will have a maximum diameter which is from 0.1 mm to 1.0 mm larger than a maximum diameter of the adjacent distal and/or proximal flanking regions, usually being the range from 0.13 mm to 0.6 mm, and often in the range from 0.15 mm to 0.5 mm.

In a preferred example, a central non-uniform region (e.g. a convex-shaped region, a dome-shaped region or other enlarged region) of the inflatable balloon has a maximum diameter which is from 0.15 mm to 0.35 mm larger than a maximum diameter of the adjacent distal and/or proximal flanking regions when the balloon is inflated to nominal pressure (or labeled), and wherein at least one of the flanking region lengths ranges from 1 mm to 6 mm, preferably from 1 mm to 4 mm, more preferably from 1 mm to 3 mm, and wherein the length from a transition point (between central region and proximal and/or distal flanking regions) to a point of maximum diameter ranges from 2 mm to 14 mm, preferably ranges from 3 mm to 10 mm, more preferably ranges from 4 mm to 8 mm, where the proximal and distal length may be the same or different depending on whether the point of maximum diameter is at or near the center of the central non-uniform region or not. The balloon is configured to deploy a stent from a crimped diameter to a deployed larger configuration wherein the largest stent diameter is located adjacent to the maximum inflatable balloon diameter, and wherein the stent after deployment by said balloon has sufficient strength to support a body lumen.

In another example, the central convex region of the inflatable balloon is larger than the distal and/or proximal flanking regions when the balloon is in the inflated condition, e.g. when the balloon is inflated to a pressure in the range from nominal (labeled) to RBP pressure, in air, in water, in water at 37° C., and/or under physiologic conditions. At least one of said distal and/or proximal flanking regions has a second flanking region having smaller diameter than said at least one distal and/or proximal flanking region. The second flanking region has a length ranging from 0.1 mm to 6 mm, preferably 1 mm to 6 mm, more preferably ranging from 1 mm to 3 mm when the balloon is in the inflated condition. The transition angle between the at least one distal and/or proximal flanking region, and second flanking region ranges from 100° to 179.5°, preferably ranges from 125° to 179°, more preferably ranges from 150° to 179°, and often within any of the ranges set forth above.

In one example of the present invention, an inflatable balloon has a central convex region having a larger diameter than an adjacent proximal flanking region, wherein the proximal flanking region has a length in the range from 0.1 mm to 5 mm, preferably from 0.5 mm to 5 mm, and wherein the transition angle between the central region and the adjacent distal flanking region ranges from 150° to 179°, preferably ranges from 160° to 179°, more preferably ranges from 170° to 179°, or in any of the other ranges set forth herein, and wherein the , when the balloon is in the inflated condition tested in air, in water, in water at 37° C., and/or under physiological conditions, wherein the diameter of a distal flanking region is substantially equal to or smaller than the diameter of the proximal flanking region, if any.

In one example of the present invention, an inflatable balloon has a central convex region having a larger diameter than an adjacent distal flanking region, wherein the distal flanking region length ranges from 0.1 mm to 5 mm, preferably ranges from 0.5 mm to 5 mm, and wherein the transition angle between the central region and the adjacent distal flanking region ranges from 150° to 179°, preferably ranges from 160° to 179°, more preferably ranges from 170° to 179°, and wherein the transition angle between the convex central region and the adjacent proximal region or point ranges from 170° to 179°, or in any of the other ranges set forth herein, and wherein the , when the balloon is in the inflated condition tested in air, in water, in water at 37° C., and/or under physiological conditions, wherein the diameter of a proximal flanking region is substantially equal to or smaller than the diameter of the distal flanking region, if any.

In another example, an inflatable balloon has a convex central region larger with a maximum diameter when inflated which is larger than at least one adjacent proximal and/or distal flanking region, wherein the transition angle between the central convex region and the adjacent flanking region(s) ranges between 179° and 179.5°, preferably ranges between 179° and 179.6°, more preferably ranges between 179° and 179.7°, most preferably ranges between 179° and 179.8°, or any of the other ranges set forth herein.

In another example, an inflatable balloon has a convex central region having a maximum diameter ranging from 0.15 mm to 0.25 mm larger than a maximum diameter of an adjacent flanking region or transition region where the convex central regions meets the flanking region(s), and wherein the convex central region transition angle to the adjacent conical ends has an angle ranging from 175° to 179.5, or any of the other ranges set forth herein.

In a preferred example, an inflatable balloon has a non-uniform shaped central region, and at least one adjacent flanking region, wherein a transition angle between the central non-uniform shaped region and the at least one flanking region ranges from 150° to 179°, preferably from 160° to 179°, and more preferably from 170° to 179°, or any of the other ranges set forth herein. The maximum central non-uniform diameter ranges from 0.15 mm to 0.35 mm larger than the largest diameter of an adjacent flanking region when the balloon is in the inflated condition, or inflated to nominal pressure, in air, in water, in water at 37° C., and/or under physiologic conditions.

In one example, a nominal diameter (or labeled diameter) is identified in an “instructions for use” which accompanies the balloon delivery catheter referring to a region on the working length of the inflatable balloon, and typically refers to the diameter of at least one of the flanking regions when inflated. In a preferred example, the nominal diameter of the inflatable balloon refers to the anticipated or to the intended reference vessel or mean reference vessel to be treated. In another example, the compliance chart of at least one of the flanking regions would be listed or graphed covering pressures ranging at least from nominal to RBP. In yet another example, the IFU would also list or graph the maximum diameter, magnitude of the convex central region, and/or location of the maximum diameter, at ranges from nominal to RBP. In yet another example, the IFU lists the compliance of the convex central region at pressures ranging from nominal to RBP. In yet another example, the product label can list one or more of the information in the IFU.

In a second aspect or example of the present invention, a stent delivery system comprises a stent delivery catheter, as in any of the examples and embodiments described above in combination with a stent positioned over the inflatable balloon of the stent delivery catheter so that the stent spans the central convex region of the balloon as well as at least a portion of at least one of the flanking regions of the balloon after the balloon is inflated or in the inflated balloon configuration or condition. Usually, but not necessarily, the stent will extend over substantially the entire lengths of the convex central region and flanking regions of the balloon after the balloon is inflated (or in the inflated balloon configuration). More usually, the stent will extend over the entire lengths of the convex central region and flanking regions of the balloon except for at least a portion of at least one of the flanking regions of the balloon ranging from 0 to 1.5 mm, after the balloon is inflated or in the inflated balloon configuration. Inflation of the balloon causes the central region of the stent to expand to an incrementally greater diameter than do adjacent proximal and/or distal regions of the stent, such proximal and/or distal regions of the stent correspond (or overlap) at least a portion of the proximal and/or distal balloon flank regions. In particular, inflation of the central convex region of the balloon will engage the central region of the stent to affect such greater differential expansion than inflation of the proximal and distal regions of the stent over the proximal and distal flanking regions of the balloon. The amount of increased differential expansion of the central region of the stent when compared to the expansion of the proximal and/or distal region of the stent will generally correspond to the differences in the inflation diameters of the central convex region and the distal and proximal flanking regions of the stent as set forth above.

In a third aspect or example of the present invention, a method of treating a vessel lesion comprises providing a catheter having an inflatable balloon with a central region, a proximal flanking region, and/or a distal flanking region. A stent is positioned over the inflatable balloon so that the stent spans the central region as well as at least a portion of at least one of the flanking regions of the balloon. The stent delivery catheter is advanced to position the stent at the vessel lesion, and the balloon is inflated to differentially expand the central region of the balloon relative to said at least one of the adjacent flanking regions. The differential inflation of the balloon regions in turn differentially expands the stent within the vessel lesion. For example, a lesser expansion of a distal flanking region of the balloon can accommodate vessel anatomy where the vessel diameter tapers in the distal direction. This reduces edge dissections while achieving optimal stent deployment, especially in the central region of the stent where usually the lesion is present. In such methods, the central region of the balloon may expand to a convex configuration relative to the at least one flanking region when inflated. At least the distal flanking region may be expanded to a diameter less than a diameter of the central region so that a distal segment of the stent is expanded less than a central segment. The central region of the balloon may be expanded to a convex configuration relative to the at least one flanking region when inflated. The stent may extend over substantially the entire length of the convex central and flanking both regions of the balloon so that each region differentially expands corresponding segments of the stent as the balloon is inflated. Such methods may further comprise deflating and removing the balloon from the stent after deployment in the vessel lesion, wherein a central segment of the stent substantially maintains a larger diameter relative to the at least one flanking region, or wherein the stent central segment will have substantially similar diameter to at least one flanking region after deployment as a result of the lesion opposite force to the stent central lesion expansion, where in the absence of having the central larger diameter segment, the stent in the central segment can become smaller in the central segment after deployment as a result of the opposite force the lesion provides against the stent expansion.

In a preferred example, the flanking region length (or proximal and/or distal adjacent stent regions) from the nominal inflated pressure to RBP remains substantially the same length. In another example, the flanking region length (or proximal and/or distal adjacent stent regions) from nominal pressure to RBP pressure decreases by 1-2 mm. In a third example, the flanking region length (or proximal and/or distal adjacent stent regions) from nominal pressure to RBP pressure maintains at least a 0.5 mm to 4 mm flanking region length. In a fourth example, at least a portion of the flanking region (or proximal and/or distal adjacent stent regions) is maintained when the stent is expanded at pressure ranging from nominal pressure to RBP pressure.

In one example, at least one of the flanking regions will have a length ranging from 0.5 mm to 8 mm, preferably ranging from 1 mm to 6 mm, more preferably ranging from 1 mm to 4 mm.

In one example, the resulting expanded profile of the stent (where a stent central region has a larger diameter than an adjacent proximal and/or distal region) will typically be substantially maintained after the balloon is deflated and removed from the stent. While there may be some degree of recoil, the inward recoil will typically be less than 10% of the stent diameter along its length or segments, usually being less than 7%, and often being less than 5%. Alternatively, the recoil ranges from 2% to 10%, preferably ranges from 2% to 7%, and more preferably ranges from 2% to 5%. The recoil of the stent, after deployment of the stent (or after expansion of the stent) from a crimped configuration to a deployed expanded configuration and then deflation of the balloon, in the stent central region (corresponding (or overlapping or adjacent) at least in part to the central convex region of the balloon) maybe different or substantially the same recoil from the adjacent proximal and/or distal regions of the stent (corresponding (or overlapping) to at least a portion of at least one of the flank regions).

In another example, at least one of the proximal and/or distal regions length of the stent adjacent to the central region of the stent becomes shorter (and/or becomes part of the stent central region where the transition angle of the stent ranges from 170° to 179°) as the stent is expanded from nominal pressure to rated burst pressure (atm). The proximal and/or distal length of the stent may become shorter at RBP (and/or becomes part of the stent central region where the transition angle of the stent is ranges from 150° to 179°, preferably from 170° to 179°) compared to length at nominal by an amount ranging from 0% to 80%, preferably ranging from 25% to 75%, more preferably ranging from 35% to 65%, when expanded in air, in water, in water at 37° C., and/or under physiologic condition. Alternatively, for example, at least one of the proximal and/or distal stent region length shortens (and/or becomes part of the larger stent central region where the transition angle of the stent ranges from 170° to 179°) by a magnitude ranging from 0 mm to 3 mm, preferably ranging from 1-2 mm, when the balloon is inflated from nominal pressure (or labeled) to RBP pressure. In one example the stent proximal and/or distal regions are substantially flat, or tapered (for example either increasing or decreasing in diameter in a direction away from the stent central region), or has a shape of less convex than the central stent region, or slight concave. In another example the stent proximal and/or distal regions length ranges from 1 mm to 8 mm preferably ranges from 1 mm to 6 mm, more preferably ranges from 1 mm to 4 mm, and most preferably ranges from 1 mm to 3 mm.

In a preferred example, the central convex region of the stent will have a length ranging from 30% to 90% of the stent length, preferably ranging from 40% to 85% of the stent length, more preferably ranging from 50% to 80% of the stent length.

In another example, the balloon convex central region extends into at least one or both of the proximal and/or distal flanking regions as the balloon is inflated (expanded) from a nominal (labeled) pressure to RBP, where the transition angle in a preferred example between the central convex region and said proximal and/or distal region is substantially maintained. In other examples, the transition angle becomes smaller. In a third example, the transition angle becomes larger. In all of the above examples, the transition angle between the central region and the at least one flanking region will be in the range of 150° to 179°, preferably ranging from 170° to 179°. In another example at least one flanking region length becomes shorter as the balloon is inflated from nominal pressure (labeled) to RBP. The at least one flanking region shortens by a range from 1, 2, 3, or 4 mm. Alternatively, the flanking region length in the above example shortens by 0.25%, 0.50%, 0.75% of the length measured from nominal pressure (labeled) to RBP. In another example, the stent exhibits the same or similar behavior and measurements as that of the balloon behavior and measurement, in this paragraph and examples. The balloon or stent are expanded in air, water, water at 37° C., and/or under physiologic condition.

In one preferred example, the delivery system is configured to have a convex central region having a maximum diameter that is larger than at least one flanking region diameter or mean diameter when the balloon is in the inflated configuration or condition, and wherein said stent has been crimped onto said delivery system balloon covering all said convex region and covering at least a portion of at least one flanking region, and wherein said convex central region expands a central region on said stent to a larger diameter (configuration) compared to a proximal and/or distal stent regions when the balloon is in the inflated configuration. The stent central region maximum diameter is larger than at least one flanking region diameter (or corresponding stent diameter) by a magnitude ranging between 0.1 mm and 1 mm, preferably ranging from 0.12 mm to 0.5 mm, most preferably ranging from 0.15 mm to 0.35 mm. In another example, a stent having a patterned structure, said structure comprising a plurality of rings, each ring is connected to an adjacent ring in at least one location, said stent having at least some struts with thickness at any point (or having a mean thickness) of ranging between 70 mm and 170 micro meters, preferably ranging between 90 mm and 150 mm. In another example said stent is biodegradable polymeric or biodegradable metallic stent. In another example said stent is substantially non degradable. The stent is expanded in air, in water, in water at 37° C., and/or in physiologic conditions. In another example, the stent comprises a patterned structure said structure comprises structural elements such as struts, crowns, and links, said structure is configured to have a substantially convex abluminal surface shape on at least some of the structural element (in a cross section view of the structural elements), preferably having a convex shape on substantially all of the stent structural elements. In another example, the stent being expandable to rated burst pressure of the balloon without fracture.

In one example, the angles, length, width, thickness, and/or other measurements are measured on the balloon mold, the balloon in the inflated condition, the balloon at nominal (or labeled pressure, or RBP pressure), and/or the stent. When measured on the stent in the expanded configuration at the nominal (labeled) pressure or RBP for example, the transition angle measurements for example can be measured utilizing one or more of the stent structural element (such as the strut, crown, or link) adjacent to the transition, the mean of one or more of the structural elements adjacent to the transition, and/or an approximation of the transition angle based on the geometry of the stent structural elements adjacent to the transition.

In one example, a balloon is formed by blowing a tube typically made from the desired material under heat and pressure within the constraints of a mold in following steps. A typical balloon forming process would be as follows: 1) The tube is extruded through a die under heat and pressure followed by quenching. 2) The tube is further drawn down by cold stretching through a die to a smaller diameter of such that a small section is left undrawn. The other side of the undrawn section is then similarly drawn down in diameter. The length of this undrawn section is dictated by the desired balloon length typically around half the length of the desired balloon working length. 3) The semi-drawn tube with its undrawn section along with drawn section on both sides are then placed inside a mold. 4) The mold is heated while the semi-drawn tube is pressurized for a short period of time during which, the tube expands and conforms to the mold. In the process the tube takes on the shape of the mold while thinning out to form into a balloon. As can be understood the balloon can be shaped as desired by shaping the mold accordingly. The mold is traditionally consisting of the two end segments and a mid-segment. The inside of the two end segments being conical forming the balloon tapers and the inside of the mid-segment forming the central contoured section of the balloon 5) The mold is then cooled and the formed balloon is removed. 6) The balloon is then attached to the catheter shaft over and folded radially into a smaller unexpanded diameter. 7) If desired, a stent is crimped over the balloon. In another example of a process of making the contoured the balloon is contoured after it is attached to the catheter. By this process the steps 1 through 6 are essentially or are similarly the same except the mold is not contoured but, has fully cylindrical transition shape. The catheter is the put though following short steps: 1) The balloon portion of a fully or substantially assembled balloon/catheter is placed in a mold having the shape of the flank regions and the contoured (convex) central region. 2) The catheter balloon is then subjected to pressure while simultaneously applying heat focused at the segment of the balloon to be contoured (convex). 3) The mold is then cooled and the balloon and catheter are then removed from the mold.

The stents delivered by the stent delivery systems of the present invention may be metal or polymeric, often being polymeric and even more often be biodegradable polymeric or metallic stents which are at greater risk of damage or fracture from the stepped balloons of the prior art. The polymeric and biodegradable polymeric stents of the present invention may be patterned from a polymer tube as described in commonly owned PCT Patent Application, PCT/US2016/026821 (Attorney Docket No. 32016-712.604), or any of the other commonly owned application previously incorporated herein by reference.

The stents delivered by the stent delivery systems of the present invention may themselves have a uniform geometry which, absent delivery by the contoured balloons of the present invention, would deploy to a substantially uniform diameter or configuration. In such cases, it is use of the contoured balloons of the present invention which will impart the desired geometries to the stents upon or after deployment by balloon inflation. In other instances, the stents may be fabricated or modified to possess a non-uniform geometry which is configured to deploy into the desired contoured stent shape when delivered by the shaped balloons of the present invention having a convex central region and flat or substantially flat flanking regions adjacent to the transition region or angle.

The stents of the present invention may be formed by known stent fabrication procedures for metal and/or polymeric stents, such as those described in commonly owned PCT Patent Application, PCT/US2016/026821 (Attorney Docket No. 32016-712.604), previously incorporated herein by reference hereinabove. For example, the stents may be formed to have known strut patterns by laser cutting, chemical etching, drawing, extrusion, spraying, printing, and/or molding, or the like. The strut patterns may be uniform or substantially uniform across the entire length of the stent or may be different for different regions of the stent, for example being different for the central convex region and either or both of the proximal and distal flanking regions. Moreover, the distal and proximal regions of the stent which are adjacent to the expanded central region may have the same or different strut patterns.

For example, the central region of the stent can have struts that are longer in length compared to struts at one or both of the stent flanking regions. The struts can be longer from a range of 0.1 mm to 1 mm, preferably from 0.2 mm to 0.75 mm. The strut thickness for example can be thicker in the central stent region (or part of it), thicker than one or both of the adjacent proximal and/or distal flanking regions strut thicknesses. The thickness increase can range from 0.01 mm thicker to 0.1 mm, preferably from 0.025 mm to 0.5 mm. The number of crowns can be larger at least in a portion of the stent central region compared to one or more of the adjacent flanking regions. The number of crowns can increase from a range of 1 to 4 crowns, preferably from a range of 1 to 2 crowns.

In other examples and embodiments of the present invention, the stent may be configured to have a uniform or substantially uniform diameter when crimped over the balloon and to acquire the desired contoured shape with a dome central region (or a substantially dome shaped central region) and flat or substantially flat proximal and/or distal adjacent regions, preferably adjacent to the central region and/or the transition angle, after deployment by the delivery balloon. Usually, a single stent will be positioned over the inflatable balloon in the deflated condition for delivery, but in other instances, multiple stents may be positioned over the inflatable balloon in the deflated condition for simultaneous delivery.

The stents may be formed by any conventional techniques, optionally being formed as slotted tubes, braided coils, braided filaments, ratcheting stent structures, and the like. Often, the central region of the stent which is expanded to a greater deployed geometry will be configured to engage a luminal stenosis or other particular anatomy in a manner which resists, reduces, and/or inhibits narrowing into an hourglass or dog bone configuration as discussed previously.

In a preferred example, the substantially cylindrical expanded stent is positioned over a non-inflated balloon with the convex central region of the balloon adjacent (under) to the central region of the stent. The stent is gradually crimped onto the un-inflated balloon using a radially uniform force and heat. Once, the desired crimped stent profile is achieved, the balloon is pressurized while still constrained by the stent which remains under the radially uniform force and heat such that the balloon does not expand. The balloon is then depressurized and the uniform radial force and heat on the stent are removed. The stent is optionally sheathed.

In another preferred example, the substantially cylindrical partially crimped stent is positioned over the un-inflated or partially inflated balloon with the convex central region of the balloon adjacent to (under) the central region of the stent, and placed in a heated crimping fixture. The heated crimping fixture is closed onto the partially crimped stent, until it contacts the stent positioned on the un-inflated or partially inflated balloon. The balloon is then pressurized while constrained by the heated crimping fixture and scaffold. The stent is gradually crimped onto the partially inflated balloon using radially uniform force and heat, while gradually depressurizing and deflating the balloon until the desired crimped stent profile is achieved. The stent is optionally cooled to below the glass transition temperature. The mounted stent is removed from the crimp fixture, and optionally sheathed.

In a third preferred example, the substantially cylindrical expanded stent is positioned over the inflated balloon with the convex central region of the balloon adjacent (under) to the central region of the stent. The stent is gradually crimped onto the inflated balloon using radially uniform force and heat, while gradually deflating the balloon until the desired crimped stent profile is achieved. The stent is optionally sheathed.

An exemplary biodegradable stent (scaffold) may be formed from or otherwise comprise a biodegradable polymeric material which may include one or more polymers selected from the group consisting of poly-L-lactide, poly-DL-lactide, polylactide-co-glycolide, polylactide-co-polycaprolactone, poly (L-lactide-co-trimethylene carbon-ate), polytrimethylene carbonate and copolymers thereof; polyhydroxybutyrate and copolymers thereof; polyhydroxy-valerate and copolymers thereof; poly orthoesters and copolymers thereof; poly anhydrides and copolymers thereof; polylactide and copolymers thereof; polyglycolides and copolymers thereof; polycaprolactone and copolymers thereof; and polyiminocarbonates and copolymers thereof; iodinated poly (desaminotyrosine carbonate); tyrosine-derived polycarbonates; tyrosine-derived polyacrylates. The biodegradable polymeric material can be a homopolymer, copolymer, graft polymer, block polymer, or a blend of two or more homopolymers and/or copolymers.

In a preferred example, it is desirable to have a degradable stent having structural elements (such as struts, crowns, and links) wherein at least some of the structural elements having thicknesses being in the range of 80 mm to 135 mm, and/or at least some of said structural elements widths being in the range from 80 mm to 170 mm, and/or at least some of said structural elements having a cross sectional area ranging from 6500 μm² to 25000 μm², and/or the degradable and/or the polymeric material substantially full degraded from 3 months to three years, preferably substantially degraded from 6 months to 2 years. However, degradable stents with such properties and/or dimension ranges can exhibit one or more of high recoil, low stent strength (sometimes not sufficient to support a body lumen), and/or fracture upon expansion of the stent to nominal or to a diameter above nominal (due to the weakness and thickness of the stent material and/or material properties such as brittleness and insufficient elongation of the material upon expansion) which can results in suboptimal procedure. The balloon delivery system of the present invention is configured to deploy a degradable stent within one or more of the ranges and/or properties above, to achieve an optimal implantation of said degradable stent, and/or achieve an optimal procedure, wherein the degradable stent alone (unaided by the balloon in the present invention but rather deployed by a conventional balloon) does not have sufficient strength to support a body lumen, has a high recoil, and/or fractures upon expansion or further expansion, wherein the degradable stent deployed by the balloon of this invention allows the stent implantation to be optimal, and/or acceptable. The inflatable balloon of this invention expands the degradable stent in a more concentric manner, and/or improves concentricity of the expanded stent.

In a preferred example, a degradable stent having properties as described in this application may be deployed by a balloon catheter of the present invention, and wherein the percentage of residual stenosis diameter or mean percentage residual stenosis diameter, as measured for example visually using x-ray, QCA (such as online QCA, offline QCA, or as commonly known or used in the art), for example in a cohort of patients ranging from 5 patients to 2000 patients or more, wherein the patients are enrolled substantially in accordance with the Instruction for Use, or in accordance with a controlled clinical study, wherein the mean percentasge diameter stenosis is measured post-deployment of the stent, and wherein the stent is expanded by the balloon of the present invention and/or re-expanded by the balloon to achieve an optimal implantation, and wherein the mean percentage diameter stenosis ranges from 5% to 18%, preferably ranges from 5% to 15%, and more preferably ranges from 5% to 13%.

In a preferred example, a degradable stent having properties as described in this application may be deployed by a balloon catheter of the present invention, and wherein the percentage residual stenosis diameter or mean percentage residual stenosis diameter (as measured for example visually using x-ray, QCA (such as online QCA, offline QCA, or as commonly known or used in the art), for example in a cohort of patients ranging from 5 patients to 2000 patients or more, wherein the patients are enrolled substantially in accordance with the Instruction for Use, or in accordance with a controlled clinical study, wherein the mean percentage diameter stenosis is measured post deployment of the stent, and wherein the stent is expanded by the balloon of the present invention and/or re-expanded by the balloon to achieve an optimal implantation, and wherein the mean % diameter stenosis ranges from 5% to 18%, preferably ranges from 5% to 15%, more preferably ranges from 5% to 13%.

In another example, a degradable or other stent deployed with a balloon according to this invention, is deployed (or expanded) to a pressure ranging from a nominal (or labeled) pressure to a RBP, wherein the stent lumen area (or mean stent area) at about the maximum expanded diameter central region of the stent is larger than the stent lumen area at a proximal and/or distal adjacent flanking regions, by a range from 0.0175 mm² to 0.12 mm², wherein the flanking region adjacent to the transition angle is substantially flat. The maximum diameter of the stent central region is larger than an adjacent proximal and/or distal adjacent flanking region diameter by a magnitude ranging from 0.15 mm to 0.5 mm. Diameters and mean areas can be measure in air, water, water at 37° C., or under physiologic conditions.

In one example, a radial strength of the stent is measured by a pressure vessel method (where for example the pressure to reduce the radial diameter of the stent by 25% is measured), by a flat plate method (where the force to reduce the diameter of the stent for example by 10% is measured), and/or by other methods known to one skilled in the art, or other in-vitro or in-vivo methods. Recoil may be measured on the bench or in-vivo as commonly known in the art. Degradation can be measured in-vitro and/or in-vivo by measuring a break-down time of the polymer chain from prior to implantation (or upon deployment) to about break down of 75%-90%, by collecting at least three data points (one months, two months, three months, or four months apart) and approximating the remainder exponential decay curve using standard scientific methods. The tests can be performed in air, water, water at 37° C., and/or under physiological conditions.

In another example, a degradable or other stent comprises is deployed to an expanded configuration in a diseased vessel (or at a lesion site), said stent having a central region having a shape that is substantially convex in the deployed configuration (e.g. a dome shape), and/or having a maximum diameter in said central region, and wherein the stent has at least one proximal and/or distal adjacent flanking regions having a substantially flat region adjacent to the transition angle (between the central region and the flanking region), said flanking region has a diameter smaller than the maximum stent diameter of the stent central region, and wherein the diameter of the stent central region after expansion becomes substantially equal to or smaller than the diameter of said flanking region.

In another example, the stent, preferably degradable stent comprises degradable metal or metal alloy such as magnesium metal or magnesium alloy.

In one example, the term central region (for the balloon or the stent) is used to refer to the region having the maximum diameter (largest diameter) of the balloon or the stent. The central region location however can be substantially in the center of the balloon or stent, can be located proximal to the center of the balloon or stent, or can be located distal to the center of the balloon or the stent.

Examples of non-degradable stent materials include but are not limited to metals and metal alloys, such as stainless steel, such as 304V, 304L, and 316LV stainless steel; steel alloys such as mild steel; cobalt-based-alloys such as cobalt chrome; L605, Elgiloy®, Phynox®; platinum-based alloys such as platinum chromium, platinum iridium, and platinum rhodium; tin-based alloys; rhodium; rhodium based-alloys; palladium; palladium base-alloys; aluminum-based alloys; titanium or their alloy; rhenium based-alloys such 50:50 rhenium molybdenum; molybdenum based-alloys; tantalum; gold and gold alloys; silver and silver alloys; shape memory metal or alloys; chromium-based alloys; nickel-titanium alloys such as linear-elastic and/or super-elastic nitinol; nickel alloys such as nickel-chromium-molybdenum alloys (e.g., INCONEL 625, Hastelloy C-22, Hatelloy C276, Monel 400, Nickelvac 400, and the like); nickel-cobalt-chromium-molybdenum alloys such as MP35-N; nickel-molybdenum alloys; tungsten and tungsten alloys; platinum enriched stainless steel; combinations thereof; or the like, and other malleable metals of a type commonly employed in stent and prosthesis manufacture.

In another example, although the balloon of the present invention is suitable for nondegradable stent in general, to further improve acute outcome, eliminate or minimize the frequency for a post dilatation balloon with a different catheter, or other; it is more required when configured to deploy a non-degradable stent having structural elements (such as struts, crowns, and links) where at least some of the structural elements having thicknesses being in the range of 40 mm to 80 mm, preferably in the range from 40 micrometer to 75 micrometer, more preferably in the range from 40 mm to 65 mm; and/or at least some of the stent structural elements having widths in the range from 40 mm to 90 mm, preferably in the range of 40 mm to 85 mm, more preferably in the range of 50 mm to 75 mm; and/or wherein at least some structural elements of the stent have cross sectional area ranging from 1500 micro-meters² to 5600 micro-meters². However, stents with such properties and/or dimension ranges can exhibit one or more of high recoil, low stent strength (sometimes not sufficient to support a body lumen), and/or fracture upon expansion of the stent to nominal or to a diameter above nominal (due to the weakness and thickness of the stent material and/or material properties such as brittleness and insufficient elongation of the material upon expansion) which can results in suboptimal procedure. The balloon delivery system of the present invention is configured to deploy non degradable stents within one or more of the ranges and/or properties above, to achieve an optimal implantation of said degradable stent, and/or achieve an optimal procedure, wherein the stent alone (unaided by the balloon in the present invention but rather deployed by a conventional or other balloon) does not have sufficient strength to support a body lumen, has a high recoil, and/or fractures upon expansion or further expansion, wherein the stent deployed by the balloon of this invention allows the stent implantation to be optimal, and/or acceptable.

In another example, the balloon delivery system of this invention is configured to perform one or more of the following: deploy (expand) a stent in a non-uniform central region (or convex) shape without edge dissections within the flanking region and/or adjacent to the flanking region, further expanding of the stent to a larger diameter without edge dissections within the flanking regions and/or adjacent to the flanking regions, and/or eliminate (or minimize) the need for a post dilatation balloon with a different balloon dilatation catheter, and/or improve acute implantation of the stent outcome, and/or to improve stent concentricity in the expanded configuration, especially in a diseased mammalian lumen).

In another example, it is desirable to implant a degradable stent, more desirable to have a degradable material that degrades in a period ranging from 3months to 2 years, preferably degrades in a time period ranging from 3 months to 18 months. However, degradable stent material can have unwanted negative effects such as inflammation and stent thrombosis after stent implantation and/or as the material degrades over time, and especially as the material degrades in a short time period from 3 months to 2 years. It is therefore desired to reduce or minimize the amount of degradable material in the body to reduce or eliminate the unwanted negative effects. It is desirable to have the degradable material weight, preferably the degradable polymeric material weight or mean weight ranging from 0.3 mg/mm of stent length to 1 mg/mm of stent length (mm of stent length such as 4.2 mg to 14 mg for 14 mm long stent, or 5.4 mg to 18 mg for18 mm long stent, etc.), preferably ranging from 0.3 mg/mm of stent length to 0.9 mg/mm of stent length. Degradable stents formed from degradable materials having weights in the range from 0.4mg/mm to 1mg/mm tend to be weaker stents compared to non-degradable stents, usually having 10% compression flat plate strength ranging from 0.1 N to 0.4 N to as high as from 0.4 and 0.7N for a 3.0 mm stent by 14 mm length, and/or usually having pressure vessel strength testing ranging from 5 psi to 15 psi to as high as from 15 psi to 23 psi, and/or fractures upon expansion to a deployed configuration. An inflatable balloon of the present invention with a central convex region having a larger diameter than at least one adjacent distal and/or proximal flanking regions, wherein the central convex region has a maximum larger diameter ranging from 0.15 mm to 0.35 mm at a nominal (or labeled) pressure compared to the maximum diameter of the at least one flanking region, and wherein the transition angle between the central region and the at least one flanking region ranges from 150° to 179.5°, preferably ranging from 160° to 179° , more preferably ranging from 170° to 179°. The inflatable balloon of the present invention allows for gentle expansion of the stent, or gradual expansion of the stent, or expanding the stent at a shallow angle, which minimizes or eliminates stent fracture upon expansion. The inflatable balloon of the present invention having a central convex region which usually oriented to oppose a vessel or lumen lesion pushing, or further opening the lesion, by expanding a central region of the stent to a larger diameter than an adjacent flanking region diameter wherein such expansion of the central region allows the opening of the vessel to a larger diameter without causing edge dissection in the flanking regions as a result of not overly expanding the stent in the flanking region compared to the central region. The stent after balloon deflation or after balloon re-inflation and deflation allows the stent to have sufficient strength to support a body lumen, or the stent has sufficient strength to support an open a substantially open lumen. The stent typically would have a % diameter stenosis post implantation ranging from zero to 18%, preferably ranging from zero to 15%, more preferably ranging from zero to 12%, when used substantially in accordance with the IFU or in accordance with a controlled clinical trial. The inflatable balloon of the present invention can also further expand the stent to a larger configuration without causing dissection or edge dissection in the flanking region or adjacent to the flanking region.

In one example, a degradable stent degradation period comprises the breaking down of the material as measure by molecular weight of the degradable material, starting from an initial molecular weight to a molecular weight that is 10% to 25% of the initial degradable material. The degradation time period can be estimated by subjecting the degradable material to in-vito or in vivo physiologic conditions such as water at 37C and typically molecular weight can be measure at at least three or more time points over several months, and then using commonly used exponential decay equations or programs to estimate the degradation time period.

In another example, a balloon having a convex (or dome) shaped region having a maximum balloon diameter when inflated compared to the adjacent flanking regions (or maximum expanded stent diameter compared to at least one adjacent flanking region) may have non-uniform, oblong, arcuate, ellipsoid, spheroid, and other shapes, geometries, and configurations.

In other examples, the balloon (and/or stent) having a transition angle from said convex region to at least one adjacent flanking regions where the transition angle ranges from 125° to 179.5°, preferably ranges from 150° to179.5°, more preferably 170° to 179°, may have one or more flanking regions which are substantially flat adjacent to said transition angle or within 1 mm from said transition angle on the flanking regions.

In one example, a stent and delivery system comprises a balloon catheter and a stent disposed on the catheter, wherein the balloon catheter has an inflatable balloon with a distal flanking region, a proximal flanking region, and a central region having a length equal to or greater than a length of the proximal and/or distal section, wherein the central region has a diameter larger than the proximal and/or distal sections in each of an uninflated configuration and an inflated configuration.

In one example, a stent delivery system comprises a balloon catheter and a stent disposed on an inflatable balloon of the catheter, wherein the inflatable balloon with a distal region, a proximal region, and a central region having a length equal to or less than a length of the proximal and distal region, wherein the central region has a diameter larger than the proximal and distal regions in each of an uninflated configuration and/or an inflated configuration.

In another example, a stent delivery system comprises a balloon catheter and a stent disposed on the catheter, wherein the balloon catheter has in inflatable balloon with a distal region, a proximal region, and a central region having a length equal to or greater than 30% of a working length of the inflatable balloon, wherein the central region has a diameter larger than the proximal and distal regions in each of an uninflated configuration and/or an inflated configuration.

In another example, a stent and delivery system comprises a balloon catheter and a stent disposed on the catheter balloon, wherein the balloon catheter has an inflatable balloon with a distal region, a proximal region, and a central larger region having a length equal to or greater than 30% of a length of the inflatable balloon, wherein the central region has a diameter smaller than the proximal and distal regions in an uninflated configuration.

In another example, a stent and delivery system comprises a balloon catheter and a stent disposed on the catheter balloon, wherein the balloon catheter has an inflatable balloon with a distal region, a proximal region, and a central larger than proximal and/or distal region, said central region having a length equal to or greater than 30% of a length of the inflatable balloon working length, wherein the central region has a diameter substantially equal to the proximal and distal regions in an uninflated configuration.

In a further example, a stent and delivery system comprises a balloon catheter and a stent disposed on the catheter, wherein the balloon catheter has an inflatable balloon with a distal region, a proximal region, and a central region having a diameter larger than the proximal and distal regions in each of an uninflated configuration and/or an inflated configuration, and wherein the balloon central region has a length equal to or greater than 30% of the stent length.

In a further example, a stent and delivery system comprises a balloon catheter and a stent disposed on the catheter, wherein the balloon catheter has an inflatable balloon with a distal region, a proximal region, and a central region having a diameter smaller than the proximal and distal regions in an uninflated configuration, and wherein the balloon central region has a length equal to or greater than 30% of the stent length.

In a further example, a stent and delivery system comprises a balloon catheter and a stent disposed on the catheter, wherein the balloon catheter has in inflatable balloon with a distal region, a proximal region, and a central region central region having a diameter larger than the proximal and distal regions in each of an inflated configuration whereby, in an uninflated configuration, the central region and portion of the distal and proximal regions each have a diameter smaller than an end diameter of the proximal and/or distal regions in an uninflated configuration, and wherein the balloon central region has a length equal to or greater than 30% of the stent length.

In a further example, a stent and delivery system comprises a balloon catheter and a stent disposed on the catheter, wherein the balloon catheter has in inflatable balloon with a distal region, a proximal region, and a central region having the same diameter in an uninflated configuration, and wherein the balloon central region has a length equal to or greater than 30% of the stent length.

In one example, a stent and delivery system comprises a balloon catheter and a stent disposed on the catheter, wherein the balloon catheter has in inflatable balloon with a distal region, a proximal region, and a central region having a diameter larger than the proximal and distal regions in each of an uninflated configuration and an inflated configuration, and wherein the stent has a central region extending over at least 30% of a length of the stent and having a diameter greater than either or both adjacent flanking regions of the stent. Optionally, the distal stent section diameter is smaller than the proximal stent diameter section when inflated to nominal pressure (or labeled pressure).

In one example, a stent and delivery system comprises a balloon catheter and a stent disposed on the catheter, wherein the balloon catheter has an inflatable balloon with a distal region, a proximal region, and a larger central region having a length equal to or greater than a length of the proximal and/or distal region, wherein the proximal region is shorter than the distal region and/or the central region is substantially offset towards the proximal region.

In one example the transition between the distal region and a larger central diameter region and/or the transition between the proximal region and a central region having a larger diameter forms an angle from 150° to 179°. In another example, at least a portion of the distal flanking region and/or the proximal flanking region are flat or substantially flat and located within 1 mm from the transition angle.

In one example the transition between the distal and the central regions and/or the transition between the proximal and the central regions is a curve that is concave, substantially concave, or has a portion that is concave. In another example, the transition between the distal and the central regions and/or the transition between the proximal and the central regions is a curve that is convex, substantially convex, convex to a lesser degree than the central section, or has a portion that is convex.

In further examples, the stent is disposed over the distal, proximal and central regions of the catheter balloon. Upon deployment the stent forms a deployed stent with a central section having a deployed diameter larger than one or more adjacent sections of the deployed stent. The central section of the stent can be larger before or after recoil of the stent after balloon deployment (or expansion).

In one example, the central region of the balloon can have a flat or substantially flat segment or portion in the convex central region of the balloon or the central region of the stent, said flat or substantially flat region length ranging from 5% to 50% of the convex central region length, preferably ranging from 15% to 40%. In another example, said flat or substantially flat segment or portion in the central convex region can have a length ranging from 1 mm to 30 mm, preferably ranging from 2 mm to 20 mm, more preferably from 3 mm to 15 mm. In another example said flat or substantially flat segment or portion is located about a middle section of the central region, proximal to a middle section of the central region, or distal to a middle section of the central region of the balloon or stent. The flat or substantially flat segment or portion may have a diameter which is the largest diameter on the central region or adjacent to the largest diameter on the central region of the balloon and/or stent.

FIG. 7 is a graph of balloon diameters of the proximal and the distal flanking regions and the central convex region at varying pressures. In FIG. 7, the proximal and distal flanking region diameters are essentially superimposed over one another, while the convex central region shows larger diameter over the entire pressure measurement range. In one example, the diameter of the central region is larger than the proximal and/or distal flanking regions by substantially the same magnitude between nominal pressure and rated burst pressure. In other examples, the magnitude decreases as the pressure increases, or the magnitude decreases as the pressure increases from nominal to rated burst pressure of the balloon.

In some examples, the stent is biodegradable. The stent can be patterned from a polymer tube and can extend over substantially the entire balloon distal, proximal and central sections.

In some examples, the catheter balloon is formed from a non-compliant orm semi-compliant balloon material. Exemplary non-compliant balloon material include but are not limited to polyethyleneterphthalate, polyamideimide copolymer, polyetherimide, polyetherketone, polyetheretherketone, polybutyleneterphthalate, polycarbonate, polyacetate, polyphthalamide, polycrylonitrile, polyarylene, polybutadiene, polyether, polyetherketones, polyimide, polyphenylenesulfide, polyphosphazenes, polyphosphonates, polysulfone, polycarbonate/polysulfone alloy, polysulfides, polsulfide, polythiophene, polyacetylene polycarbonates, polyphenylene ether, polyetherketones, polyimide, polyphenylene, Polycarbonate/polybutylene terephthalate alloy, ABS/PC blend, carbon reinforced composites, aramid fiber reinforced composites, poly [(R)-3-hydroxybutyrate-co-8%-(R)-3-hydroxyvalerate](P(3HB-co-8%-3HV)fibers composites, liquid crystal fibers composites, and/or combinations, blends, and copolymers, polyamide (nylon 12, nylon 11, nylon 6-12, nylon 6-11, nylon 6-6, nylon 6,), nylon blends, nylon copolymers, polyetheramide copolymer, polyurethane, polyesterpolyurethane, poycarbonatepolyurethane, polyetherpolyurethane, polyolefinpolyamide, polyacrylonitrile, polytrimethyleneterephthalate, polyacrylonitrilebutadienestyrene, polyphenylsufone, polyphthalamide, polyaryletherketone, polyethersulfone, polybutyleneadipate, polyacetate, polyacrylate, ABS/Nylon blends, polycrylonitrile, and polyanhydride, polyarylene.

In some examples, the inflatable balloon is a semi-compliant or a non-compliant balloon having a substantially uniform wall thickness without additional layers of material, restraining or limiting stent members.

In one example, the balloon is expanded to initially deploy the stent or other scaffold and is then deflated. The balloon is then re-inflated within the previously deployed stent preferably at higher pressure than an initial deployment pressure. In one example, an instruction for use provided with the product includes these instructions. The balloon can remain stationary (no movement proximal or distal from the first inflation of the balloon) before the re-inflation of the balloon. Alternatively, the balloon can be repositioned to place (position) the central region proximally or distally to the first inflation position, before re-inflation of the balloon.

In one example, the stent has a uniform geometry, e.g. arrangement and dimensions of the structural elements of the stent, which absent the enlarged central region on the deployment balloon would deploy to a substantially uniform stent diameter.

In another example, the stent geometry, e.g. arrangement and dimensions of the structural elements of the stent, differs between a central section of the stent and one or more adjacent proximal and/or distal sections. The stent can have a non-uniform geometry or structural elements dimensions designed to deploy into a substantially convex or non-uniform stent shape with the central section being deployed to the larger diameter than at least one of the ends sections of the stent. In a preferred example, the transition angle ranges from 150° to 179.5°, preferably from 170°-179°. In another preferred example, the proximal and/or distal sections are flat or substantially flat at least a portion of the flanking region adjacent to the transition angle.

In another example, the stent has a proximal segment or section, a central segment or section that is different from the proximal segment pattern, and a distal segment or section pattern which is substantially the same or different than the proximal segment pattern. Alternately, the stent has a proximal segment pattern, a distal segment pattern and a central segment pattern different from at least one of the proximal segment pattern and/or a distal segment pattern.

In one example, the balloon retains the substantially convex shape at substantially all inflation pressures. In other examples, the balloon has the substantially convex shape at a nominal inflation pressure, rated burst pressure, and/or from nominal to rated burst pressures.

In another example, the balloon has a substantially convex shape at a nominal inflation pressure and a more convex shape or a less convex shape at rated burst pressure or higher.

In some examples, the balloon central section has a substantially convex shape when viewed in a longitudinal cross section along the length of the catheter. The balloon can have the substantially convex shape when inflated, when deflated, and/or both when inflated and deflated.

In some examples, upon or after deployment in air, water, or water at 37° C., the stent forms a deployed stent with a central section having a larger diameter than at least one adjacent end section of the deployed stent. In other example, upon deployment or after deployment (or expansion) of the stent in a body lumen (or physiologic conditions), the deployed stent has a central section having a larger diameter than at least one of the adjacent end sections of the deployed stent.

In one example, the inflatable balloon central region has a length equal to or greater than 20%, 30% 40%, 50% or 60% of a length of the inflatable balloon, typically measured between the connection points to the catheter shaft and subtracting the conical segments. The inflatable balloon central region preferably has a length of at least 2 mm, 3 mm, 4 mm, 5 mm, 6 mm, 7 mm, or 8 mm. In another example, the inflatable balloon central section further has a flat or substantially flat section along the length of the balloon central section, further extending over the length of the central region by at least a distance from 1 mm to 30 mm, preferably by a distance from 1 mm to 20 mm, most preferably by a distance from 1 mm to 15 mm.

In another example, the central region of the balloon is larger than the distal and/or proximal flanking regions when inflated to pressures from 1 to 40 atm, 1 to 30 atm, 1 to 20 atm, 1 to 10 atm, 6 atm to 20 atm, 10 atm to 20 atm, or any other pressure up to the nominal pressure or to the RBP, in air, water, water at 37° C., and/or physiological conditions. Preferably the central region is larger throughout the inflation pressures. In other examples, the central section is also larger in the un-inflated balloon condition.

In some examples, the central region has a longitudinal cross-sectional shape selected from the group consisting of convex, domed, tapered, pointed, flat, stepped, ribbed, ridged, pear, wave, or combinations thereof. The central section can be symmetrical or asymmetrical. The central region has a transition angle ranging from 125°-179.5° degrees, preferably ranging from 150° to 179.5° degrees, more preferably ranging from 170° to 179° degrees.

In some examples, the central region has a varying diameter along its length. In other embodiments, the central region has a substantially constant diameter along at least some of its length.

In some examples, the central convex region has a substantially constant diameter segment along a portion of the length of the central convex region. The substantially constant diameter segment can be a flat or substantially flat relative to other segments of the central convex region.

In another example, a maximum diameter of the central convex region is from 0.1 mm to 1.0 mm larger, usually from 0.12 mm to 0.5 mm larger, often from 0.15mm to 0.35 mm larger, and sometimes from 0.20 mm to 0.35 mm larger than the diameter of the distal and/or proximal flanking regions.

In another embodiment either the proximal region or the distal region or both can have variable diameter in inflated state at all pressures. The diameter can be constantly increasing or decreasing from start to end of the distal region and of the proximal region.

In another example, either the proximal region, the distal region, or both regions of the balloon can have a variable diameter in inflated state at some or all pressures. The variable diameter can be constantly increasing or decreasing from start to end of the distal and/or of the proximal region.

In another example, either the proximal region, the distal region, or both regions of the balloon can have variable diameter below nominal inflation pressuer, at nominal inflation pressure, or between nominal inflation pressure and RBP, and can have a substantially constant diameter at or above any of these pressures.

In another example, either the proximal region, the distal region, or both regions of the balloon can have a contoured shape with a maximum diameter substantially smaller than the maximum diameter of central region of the balloon.

In some examples, the central convex region, the distal flanking region, and the proximal flanking region have substantially the same or a similar compliance. In other examples, the balloon regions can have compliances which differ by an amount of 2%, 4%, 5%, 10%, or more. The balloon wall thickness can be substantially the same or different in the central convex region, the distal flanking region, and/or the proximal flanking region of the inflatable balloon. Upon deployment the stent central section can maintain a larger diameter than the adjacent end sections after removal of the balloon.

In a further example, the stent has a stent geometry, e.g. the arrangement and dimensions of the stent structural elements, configured to create a uniform shape, or non-convex shape of the stent when crimped onto the catheter and a convex shape or non-uniform shape when deployed by inflation of the balloon catheter.

In another example, the stent deployed by a balloon having a central convex region, has an initial length in a crimped configuration, wherein the length substantially is the same at nominal pressure. In another example, the initial length is measured at nominal pressure wherein the length at RBP remaining substantially the same. In a third example, the stent length at RBP becomes shorter than nominal or than the crimped configuration length by a magnitude ranging from 1% to 15%, preferably ranging from 1% to 10%, more preferably ranging from 1% to 5%.

In some examples, the stent has a proximal stent geometry, e.g. the arrangement and dimensions of the stent proximal structural elements, a central stent geometry, e.g. the arrangement and dimensions of the stent central structural elements, which is the same or different from the proximal stent geometry, and a distal stent geometry, e.g. the arrangement and dimensions of the stent distal structural elements, which is substantially the same as the proximal stent geometry.

In some embodiments, the stent has a proximal strut pattern, a central strut pattern different from the proximal strut pattern, and a distal strut pattern substantially the same as the proximal strut pattern.

In another example, the balloon catheter includes a single stent or a plurality of stents. The stent or stents are each expandable from a crimped configuration to a deployed larger configuration. In some examples, stent in the crimped configuration is substantially cylindrical and the stent is in the deployed configuration is non-cylindrical. In the non-cylindrical deployed configuration the ends of the stent have a diameter smaller than the diameter of a central section of the stent. The non-cylindrical deployed configuration has a larger diameter about a midsection of the sent. At least one proximal or distal flanking section of the stent is adjacent to a central section. The flanking section(s) forms a transition section with the central section having an angle in a range from 150° to 179.5° , preferably from 170° to 179°. The flanking section in one example is a substantially flat stent segment adjacent to the transition section.

In a further example, the balloon catheter can include a plurality of stents placed in series along the length of the balloon with substantial overlapping ends. The stents are expandable from a crimped configuration to a deployed larger configuration. In some examples, stents in the crimped configuration are substantially cylindrical. The stents in the distal and proximal sections are deployed in a either cylindrical or non-cylindrical configuration. In the non-cylindrical deployed configuration the ends of the distal and proximal stents have a diameter smaller than the diameter of a central section of the stent. The stents in the mid-section have a cylindrical configuration having diameter larger than the distal and/or proximal stent ends.

In some examples, stent is balloon deployable. In other embodiments, the stent is a combination of balloon deployable and self-expandable.

In another example, the balloon shape corresponds to the non-cylindrical shape of the stent.

In some examples, the stent is formed as a slotted tube. In other embodiments, the stent is formed from a coil or filament.

In further examples, the central region is configured to deploy the stent in opposition to a luminal stenosis.

In some examples, the stent length is from 0 mm to 3 mm, preferably from 0.1 mm to 2 mm, most preferably from 0.1 mm to 1mm, shorter than the working length of the balloon, in the crimped diameter or the inflated diameter such as at nominal pressure.

In other examples, the stent has an inner diameter which upon expansion is substantially the same as the catheter outer diameter, within 0.025 mm to 0.5 mm of the catheter balloon outer diameter, or between 0 and 0.1 mm larger than the balloon outer diameter.

In other example, the stent inner diameter when expanded is substantially the same as the catheter outer diameter, within 0.025 mm to 0.5 mm of the catheter outer diameter, or less than 0.1 mm smaller than the balloon outer diameter.

In another example, a delivery system comprises a delivery catheter for delivery of a stent, wherein the delivery catheter has a balloon with distal and proximal radiopaque marker and in addition has a central radiopaque marker corresponding to a central balloon region.

In another example, a delivery system comprises a delivery catheter for delivery of a stent, wherein the delivery catheter has distal and proximal radiopaque marker, a central radiopaque marker, and one marker under each of the distal and proximal sections of the stent and/or under the balloon flanking regions.

In another example, a method of treating a vessel lesion comprises implanting a biodegradable stent using a balloon catheter, wherein a central region of the balloon catheter has a diameter larger than adjacent flanking section(s) in an inflated configuration, wherein the biodegradable stent has a reference diameter (e.g. a labeled or nominal diameter) corresponding to an anticipated diameter of the reference vessel. The stent is advanced to the lesion using the balloon catheter, and the balloon is inflated until a central region of the balloon is larger than either or both the adjacent flanking regions. The balloon may be inflated and/or re-inflated until a central section of the stent is visibly larger than either or both adjacent stent sections by 5%, 10%, 0.1 mm, 0.15 mm, or 0.25 mm. Visual assessment may be may by any conventional imaging technology, such as fluoroscopy, IVUSD, OCT, QCA, or x-ray.

In another example, a stent delivery system comprises a balloon catheter for delivery of a self-expanding stent which has a uniform diameter along its length when crimped and which self-expands to a non-uniform geometry in the deployed configuration. The stent in the non-uniform deployed configuration has a central section with a diameter larger than adjacent proximal and/or distal end sections of said stent, preferably having convex shape over the middle section thereof.

In some examples, the stent is positioned within the catheter for delivery to the lumen and is deployed from inside the catheter in the lumen.

In some examples, the stent has a proximal strut pattern, a central strut pattern different from the proximal strut pattern and a distal strut pattern substantially the same as the proximal strut pattern.

In another example, a stent delivery system comprises a balloon catheter and a stent disposed on the catheter. The catheter is a balloon catheter having a balloon with a substantially uniform diameter along a portion of the balloon which receives the stent. The stent has a stent geometry configured to create a uniform shape of the stent when crimped onto the catheter and a convex shape when deployed by inflation of the balloon catheter with a central stent section having a diameter greater than adjacent proximal and/or distal stent sections. The stent can have a proximal strut pattern, a central strut pattern different from the proximal strut pattern and a distal strut pattern substantially the same as the proximal strut pattern.

In one example, the balloon can be made by forming balloon in a mold that has two conical end segments with extended cylindrical segments. The cylindrical portion within the end mold segment forms the flanking regions at the end of the balloon working length. The mid mold segment is split into two perfectly mating halves such that each half has inner surface in the shape of the balloon contour of the central region. The mold can also be made of two segments split in the middle, each segment consisting of the end conical shape, the flank, and the half dome-shaped contour.

In another example, the balloon contour is formed after the balloon catheter is substantially fully assembled or after the balloon has been placed onto and attached to the delivery catheter. The balloon of the substantially assembled catheter is placed in a mold having the shape of the central convex region (or other non-uniform region) and flanking regions. The balloon is then subjected to pressure while simultaneously applying heat focused at the segment of the balloon to be convex. The balloon and catheter are then removed from the mold.

One of skilled in the art would appreciate that the above examples and/or embodiments can be combined in whole or in parts without departing from the present invention.

The following numbered clauses describe other examples, aspects, and embodiments of the inventions described herein:

36. A stent delivery system comprising: a stent delivery catheter as described elsewhere herein, a stent positioned over the inflatable balloon so that the stent will span the central convex region as well as at least a portion of at least one of the flanking regions of the balloon; and inflation of the balloon to expand the stent over its entire length, wherein a central region of the stent is expanded to an incrementally greater diameter by the central convex region of the balloon than are proximal and/or distal regions of the stent by the proximal and distal flanking regions of the balloon after the balloon is inflated.

37. A stent delivery system as in clause 36, wherein the stent extends over substantially the entire length of the convex central and flanking regions of the balloon after the balloon is inflated.

38. A stent delivery system as in clause 36, wherein balloon deployment of the stent causes the central region of the stent to have a deployed diameter larger than the deployed diameter of either of the flanking regions.

39. A stent delivery system as in clause 38, wherein after removal of the deployment balloon, the stent central region of the stent maintains the larger diameter relative to the diameters of the adjacent proximal and distal regions.

40. A stent delivery system as in clause 36, wherein the flanking regions each extend beyond the ends of the stent by a length in the range from 1 mm to 6 mm.

41. A stent delivery system as in clause 36, wherein the diameters of the flanking sections on the stent are the same.

42. A stent delivery system as in clause 36, wherein the diameters of the flanking sections on the stent are different.

43. A stent delivery system as in clause 36, wherein the lengths of the flanking sections on the stent are the same.

44. A stent delivery system as in clause 36, wherein the diameters of the flanking sections on the stent are different.

45. A stent delivery system as in clause 36, wherein the flanking sections on stent are flat, substantially flat, tapered, concave, or convex.

46. A stent delivery system as in cla 33, wherein after removal of the deployment balloon, the expanded diameters of the central and flanking regions of the stent remain substantially unchanged.

47. A stent delivery system as in clause 30, wherein the stent is biodegradable.

48. A stent delivery system as in clause 35, wherein the stent is patterned from a polymer tube.

49. A stent delivery system as in clause 30, wherein the stent has a uniform geometry which absent the contoured balloon would deploy to a substantially uniform diameter.

50. A stent delivery system as in clause 30, wherein the stent has a non-uniform geometry configured to deploy into a contoured stent shape similar to that of the balloon with a convex central region and flat flanking regions.

51. A stent delivery system as in clause 30, wherein the central region has a strut pattern different from the strut patterns of the proximal and distal flanking regions.

52. A stent delivery system as in clause 39, wherein the strut pattern as in the distal flanking region is substantially the same as the strut pattern of the proximal flanking region.

53. A stent delivery system as in clause 30, wherein the stent is configured to have a uniform diameter when crimped over the balloon of the catheter and a contoured shape with a convex central region and flat proximal and distal adjacent regions deployed by inflation of the balloon.

54. A stent delivery system as in clause 30, wherein a single stent is positioned over the inflatable balloon.

55. A stent delivery system as in clause 30, wherein multiple stents are positioned over the inflatable balloon.

56. A stent delivery system as in clause 30, wherein the stent is formed as a slotted tube.

57. A stent delivery system as in clause 30, wherein the stent is formed from a coil or filament.

58. A stent delivery system as in clause 45, wherein the central region is configured to deploy the stent in opposition to a luminal stenosis.

59. A method of treating a vessel lesion comprising:

providing a stent delivery system as in any one of clauses 27-43;

advancing the stent delivery catheter to position the stent at the vessel lesion; and inflating the balloon until the central region of the balloon expands to a diameter greater than that of the adjacent flanking regions.

60. A stent delivery catheter comprising:

a catheter having an inflatable balloon; and

a stent disposed on the inflatable balloon;

wherein the inflatable balloon has a central convex region, distal and proximal end regions, and distal flanking regions between the central convex region and the distal and proximal end regions, wherein the central convex region has a length equal to at least 30% of the combined lengths of the distal flanking regions and the central convex region and wherein the central convex region has a maximum diameter larger than maximum diameters of the proximal and distal flanking regions in their inflated configurations.

61. A method of treating a vessel lesion comprising:

providing a biodegradable stent on an inflatable balloon of a catheter, wherein a central region of the inflatable balloon has a diameter larger than that of adjacent flanking regions when the balloon is inflated, wherein the stent has a labeled diameter corresponding to an anticipated diameter of the vessel and the central section of the balloon catheter has a nominal inflated diameter greater than the labeled diameter of the stent;

advancing the stent to the lesion with the balloon catheter; and

inflating the balloon until the central section of the balloon is larger than the adjacent sections.

providing a catheter having (1) an inflatable balloon with a central region, a proximal flanking region, and a distal flanking region and (2) a stent positioned over the inflatable balloon so that the stent spans the central region as well as at least a portion of at least one of the flanking regions of the balloon;

advancing the stent delivery catheter to position the stent at the vessel lesion; and inflating the balloon to differentially expand the central region of the balloon expands relative to said at least one of the adjacent flanking regions;

wherein the differential expansion of the balloon differentially expands the stent within the vessel lesion.

62. A method as in clause 62, wherein the central region of the balloon expands to a convex configuration relative to the at least one flanking region when inflated.

63. A method as in clause 62, wherein at least a distal flanking region is expanded to a diameter less than a diameter of the central region so that a distal segment of the stent is expanded less than a central segment.

64. A method as in clause 62, wherein the central region of the balloon expands to a convex configuration relative to the at least one flanking region when inflated.

65. A method as in clause 63, wherein the stent extends over substantially the entire length of the convex central and flanking both regions of the balloon so that each region differentially expands corresponding segments of the stent as the balloon is inflated.

66. A method as in clause 63, further comprising deflating and removing the balloon from the stent after deployment in the vessel lesion, wherein a central segment of the stent maintains a larger diameter relative to the at least one flanking region.

67. A stent delivery catheter comprising

a catheter body having a proximal end, a distal end, and a longitudinal axis; and an inflatable balloon on the catheter body near the distal end; said balloon having a central region, and at least one flanking region distal or proximal to said central region;

wherein the central region has a maximum diameter region which is larger than the at least one flanking region diameter by a range from 0.1 mm to 0.35 mm, and wherein the maximum central region diameter is located along the longitudinal axis at a distance from a transition angle α region ranging from 2 mm to 10 mm, and wherein the central region joins the at least one flanking region along the longitudinal axis at the transition angle α ranging from 170° to 179° in the inflated balloon configuration.

68. A stent delivery catheter comprising

a catheter body having a proximal end, a distal end, and a longitudinal axis; and an inflatable balloon on the catheter body near the distal end; said balloon having a central region, and at least one flanking region distal or proximal to said central region;

wherein the central region has a maximum diameter region which is larger than the at least one flanking region diameter by a range from 0.1 mm to 0.35 mm, and wherein the maximum central region diameter is located along the longitudinal axis at a distance from a transition angle α region ranging from 2 mm to 10 mm, and wherein the central region joins the at least one flanking region along the longitudinal axis at the transition angle α ranging from 170° to 179° in the inflated balloon configuration.

69. A stent delivery catheter as in clause 68, wherein the central region shape is spheroidal or ellipsoidal surface when inflated.

70. A stent delivery catheter as in clause 68, wherein the balloon central region has two flanking regions, one proximal and one distal to said central region.

71. A stent delivery catheter as in clause 68, wherein the distance to the maximum central region diameter along the longitudinal axis from the transition angle α ranges from 3 mm to 8 mm.

72. A stent delivery catheter as in clause 68, wherein the distance to the maximum central region diameter along the longitudinal axis from the transition angle α ranges from 4 mm to 7 mm.

73. A stent delivery catheter as in clause 68, wherein at least one of the flanking regions length ranges from 1 mm to 8 mm.

74. A stent delivery catheter as in clause 68, wherein the at least one of the flanking regions length ranges from 2 mm to 6 mm.

75. A stent delivery catheter as in clause 68, wherein at least one of the flanking regions length ranges from 3 mm to 8 mm.

76. A stent delivery catheter as in clause 68, wherein there are two flanking regions one proximal and one distal and wherein the flanking region lengths range from 1 mm to 8 mm.

77. A stent delivery catheter as in clause 68, wherein the balloon has a working length comprising the central region, the proximal flanking region, and the distal flanking region, and wherein the balloon working length ranges from 15 mm to 42 mm.

78. A stent delivery catheter as in clause 68, wherein the balloon has a labeled (or anticipated) inflation diameter ranging from 2.5 mm to 4.0 mm.

79. A stent delivery catheter as in clause 68, wherein the balloon central region has a substantially flat region, wherein the substantially flat region length ranges from 1 mm to 15 mm.

80. A stent delivery catheter as in clause 68, wherein the working length of the balloon comprises the central region and the at least one flanking region of the balloon.

81. A stent delivery catheter as in clause 68, wherein the working length of the balloon comprises the central region, the proximal flanking region, and the distal flanking regions of the balloon.

82. A stent delivery catheter as in clause 68, wherein the diameter of the proximal flanking region is substantially the same as the diameter of the distal flanking region.

83. A stent delivery catheter as in clause 68, wherein the diameter of the proximal flanking region is larger than the diameter of the distal flanking region by a range from 0.05 mm to 0.2 mm.

84. A stent delivery catheter as in clause 68, wherein the distance to the maximum central region diameter along the longitudinal axis from the transition angle α ranges from 3 mm to 8 mm.

85. A stent delivery catheter as in clause 68, wherein the distance to the maximum central region diameter along the longitudinal axis from the transition angle α ranges from 3 mm to 8 mm.

86. A stent delivery catheter as in clause 68, wherein the distance to the maximum central region diameter along the longitudinal axis from the transition angle α ranges from 3 mm to 8 mm.

87. A stent delivery catheter as in clause 69, wherein the spheroidal or ellipsoidal surface is uniformly curved between the proximal and distal flanking regions.

88. A stent delivery catheter as in clause 69, wherein the spheroidal or ellipsoidal surface has a greater curvature near its proximal and distal regions where the central region of the balloon meets the flanking regions.

89. A stent delivery catheter as in clause 68, wherein the central region has a convex shape that remains substantially from nominal pressure to RBP pressure, and wherein the at least one flanking region substantially maintains the shape at the same pressure ranges.

90. A stent delivery catheter as in clause 68, wherein a surface of the convex central region is smooth when inflated.

91. A stent delivery catheter as in clause 68, wherein a surface of the convex central region is textured when inflated.

92. A stent delivery catheter as in clause 68, wherein the at least one flanking region is generally cylindrical.

93. A stent delivery catheter as in clause 68, wherein the at least one flanking regions taper in diameter in a direction away from the central region, wherein a taper angle β of the flanking regions is less than a junction angle γ of the central convex region.

94. A stent delivery catheter as in clause 68, wherein the inflatable balloon is formed at least in part from a non-compliant material.

95. A stent delivery catheter as in clause 94, wherein the non-compliant material is selected from the group, consisting of polyethyleneterphthalate, polyamideimide copolymer, polyetherimide, polyetherketone, polyetheretherketone, polybutyleneterphthalate, polycarbonate, polyacetate, polyphthalamide, polycrylonitrile, polyarylene, polybutadiene, polyether, polyetherketones, polyimide, polyphenylenesulfide, polyphosphazenes, polyphosphonates, polysulfone, polycarbonate/polysulfone alloy, polysulfides, polsulfide, polythiophene, polyacetylene polycarbonates, polyphenylene ether, polyetherketones, polyimide, polyphenylene, Polycarbonate/polybutylene terephthalate alloy, ABS/PC blend, carbon reinforced composites, aramid fiber reinforced composites, poly [(R)-3-hydroxybutyrate-co-8%-(R)-3-hydroxyvalerate](P (3HB-co-8%-3HV)fibers composites, liquidcrystal fibers composites.

96. A stent delivery system catheter as in clause 68, wherein the inflatable balloon is formed at least in part from a semi-compliant material.

97. A stent delivery catheter as in clause 96, wherein the semi-compliant material is elected from the group consisting of polyamide (nylon 12, nylon 11, nylon 6-12, nylon 6-11, nylon 6-6, nylon 6,), nylon blends, nylon copolymers, polyetheramide copolymer, polyurethane, polyesterpolyurethane, poycarbonatepolyurethane, polyetherpolyurethane, polyolefinpolyamide, polyacrylonitrile, polytrimethyleneterephthalate, polyacrylonitrilebutadienestyrene, polyphenylsufone, polyphthalamide, polyaryletherketone, polyethersulfone, polybutyleneadipate, polyacetate, polyacrylate, ABS/Nylon blends, polycrylonitrile, polyanhydride, polyarylene.

98. A stent delivery catheter as in clause 68, wherein the central region and distal and proximal flanking regions have substantially the same compliance.

99. A stent delivery catheter as in clause 68, wherein the inflatable balloon has a single substantially uniform wall thickness.

100. A stent delivery catheter as in clause 68, wherein the inflatable balloon has a non-uniform wall thickness.

101. A stent delivery catheter as in clause 100, wherein the central region has a convex shape central region which is thinned relative to other portions of the balloon to cause the convex inflation geometry.

102. A stent delivery catheter as in clause 68, wherein the inflatable balloon is free from additional layers of material such as restraining or limiting members.

103. A stent delivery catheter as in clause 68, wherein the inflatable balloon includes additional layers of material such as restraining or limiting members to define the convex geometry of the central region.

104. A stent delivery catheter as in clause 68, wherein the balloon has a convex central shape and wherein the central region retains the convex central region and adjacent at least one flanking region at substantially all pressures ranging from nominal (or labeled) to RBP.

105. A stent delivery catheter as in clause 68, wherein a stent is placed over the balloon spanning the central region and at least in part the at least one flanking region, and wherein the stent in the expanded configuration assumes the shape of the balloon central region and the at least one flanking region.

106. A stent delivery catheter as in clause 68, wherein a stent is placed over the balloon spanning the central region and at least in part the at least one flanking region, and wherein the stent in the expanded configuration retains the shape of the balloon central region and the at least one flanking region after deployment.

### BRIEF DESCRIPTION OF THE DRAWINGS

FIGS. 1A and 1B illustrate a stent delivery catheter carrying a stent on an inflatable balloon constructed in accordance with the principles of the present invention, with the balloon uninflated (FIG. 1A) and inflated (FIG. 1B).

FIGS. 1C through 1E are cross-sectional views taken along lines 1C-1C, 1D-1D, and 1E-1E in FIG. 1B, respectively.

FIGS. 1F and 1G illustrated spheroidal and ellipsoidal shapes that may be incorporated into the inflatable balloon structures of the present invention.

FIG. 2 illustrates exemplary dimensions and angular relationships of different surface regions of the stent delivery balloons of the present invention when inflated.

FIGS. 2A through 2H are detailed views of different examples of transition regions between a central convex region and adjacent flanking regions of the stent delivery balloons of the present invention when inflated.

FIG. 3 illustrates an exemplary overlap dimension for a stent over a stent delivery balloon of the present invention.

FIGS. 4A through 4D illustrate exemplary inflatable balloon structures of the present invention with convex regions having alternative longitudinal profiles.

FIGS. 5, 5A, 5B-1, 5B-2, 5C, 5D-1, and 5D-2 illustrate exemplary surface textures that may be applied to the convex and other regions of the inflatable balloons of the present invention.

FIGS. 6A through 6F illustrate exemplary inflatable balloon structures of the present invention with flanking regions having alternative surface features and longitudinal profiles.

FIG. 7 is a diameter vs. pressure graph for the various regions of an exemplary balloon in accordance with the principles of the present invention.

### DETAILED DESCRIPTION OF THE INVENTION

The following terms and phrases as used herein and the specification and claims are defined as follows. The term “stent” refers to any implantable prosthesis, scaffold, graft, or other tubular supporting structure of the type used for maintaining patency in a human or animal body lumen, typically in an arterial lumen or a venous lumen, but also including other body lumens, such as the urinary tract, sinuses, intestinal tract, or the like. Usually, the stents of the present invention will be balloon-expandable, usually being malleable so that they may be expanded from a narrow diameter configuration to a large or extended diameter configuration. In other instances, however, this stent may be partially self-expanding, e.g. a portion of the stent structure may be formed from a self-expanding metal or polymer while the remainder is formed from a balloon-expandable material.

The term “balloon” refers to an inflatable component of a catheter which carries the stent and which expands the stent when inflated. The balloons of the present invention may be substantially non-compliant or non-distensible, in which case the balloon will be configured to be inflated to a generally fixed diameter over a wide range of inflation pressures. Usually such noncompliant or non-distensible balloons will not expand beyond 10% over their nominal inflation diameter even when inflated at pressures much higher than their intended inflation pressure. In other instances, the balloons of the present invention may also be semi-compliant, in which case the balloons may be inflated to an initial nominal diameter at a first inflation pressure while expanding to a somewhat greater diameter upon further inflation to a higher pressure, typically expanding in the range from 10% to 30% when inflated beyond the initial nominal inflation pressure.

The term “shaft” and “catheter body” are used interchangeably and both refer to the elongate structure which carries the inflated balloon at or near its distal end. The length, diameter, and other dimensions of the catheter body will be selected based upon the intended use of the catheter, e.g. in the coronary vasculature, the peripheral vasculature, the urinary tract, the intestinal tract, the sinuses, and the like. The design and construction of particular catheter bodies toward different intended uses is well known in the art and need not be described further herein.

The terms “proximal” and “distal” refer to directions along the catheter body or shaft. In particular, proximal refers to the direction of the end of the catheter body or shaft which remains outside of the patient and which is manipulated by the user during the stent placement procedures described herein. In contrast, the term “distal” refers to the direction of the end of the shaft remote from the user which is at the leading end of the catheter which is inserted into the patient lumen.

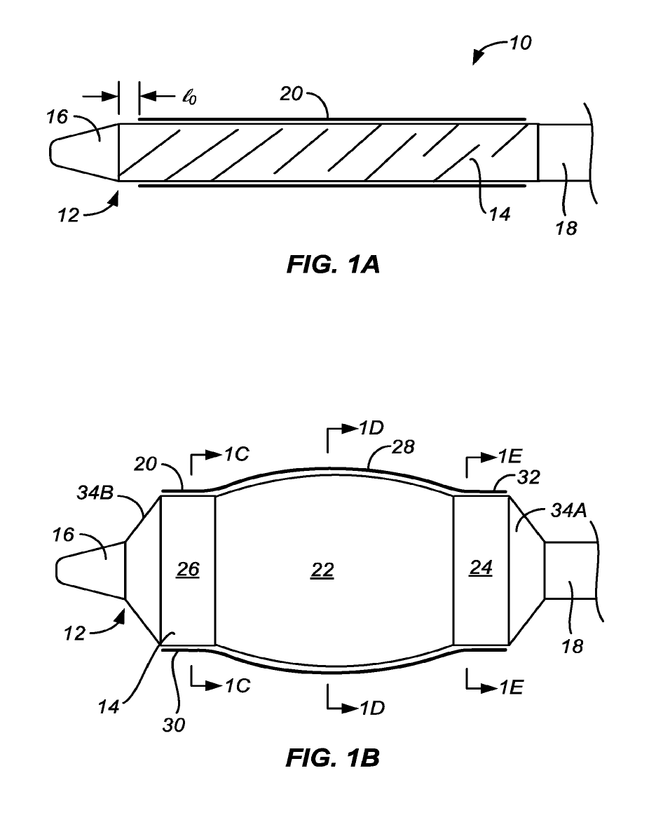

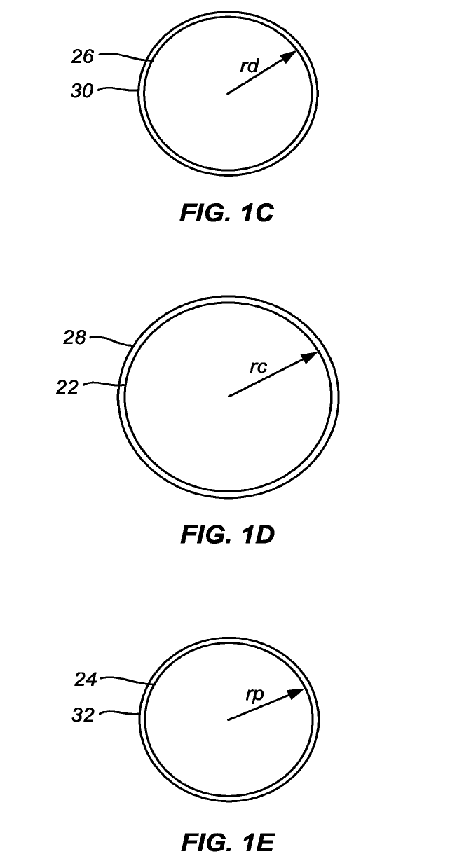

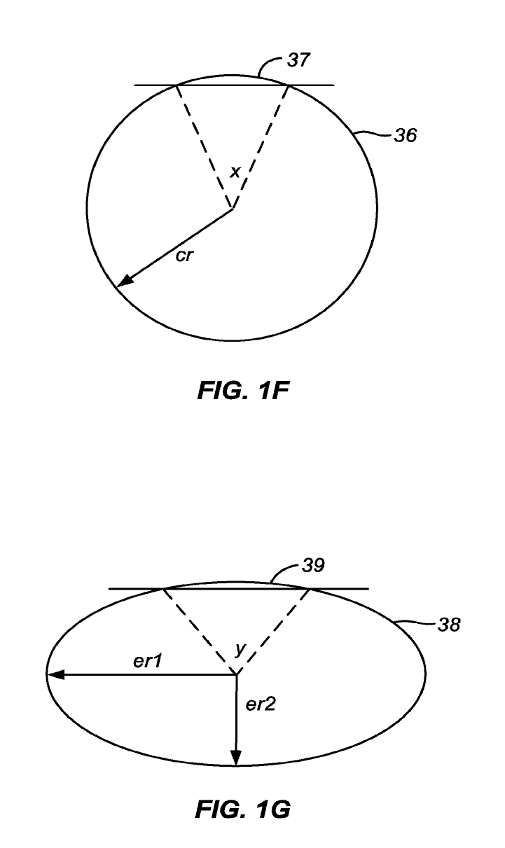

Referring now to FIGS. 1A through 1E, a stent delivery system 10 includes as its principal components a stent delivery catheter 12 and a stent 20. The stent 20 is carried over an inflatable balloon 14 at the distal end 16 of a catheter body 18. As shown in FIG. 1A, the inflatable balloon 14 is in its non-inflated state with the stent 20 thereover. The stent 20 is in its crimped, non-expanded configuration, and the stent delivery catheter is ready for introduction into a body lumen, typically a vascular lumen for treatment of a vascular lesion. The stent delivery catheter may be delivered in a variety of conventional ways, typically over a guidewire after completion of a balloon angioplasty treatment.

As shown in FIG. 1B, upon inflation, the inflatable balloon 14 displays a contoured or dome-shaped geometry with a central convex region 22, a proximal flanking region 24, and a distal flanking region 26. The stent 20 usually has a cylindrical geometry when crimped prior to expansion, but will form or assume a dome-shaped center section 28 with generally cylindrical adjacent distal and proximal sections 30 and 32 as a result of the forces applied by inflation of the contoured balloon.

Referring now to FIGS. 1C through 1E, the cross sections of the inflatable balloon 14 noted on FIG. 1B show that the distal flanking region 26 will have a circular periphery with a diameter of rd, the central convex region 22 will also have a circular periphery with a maximum radius rc, and the proximal flanking region 24 will have a circular periphery with a radius rp. In the example illustrated in FIG. 1B, the flanking regions 24 and 26 will each have substantially the same radius and will generally be cylindrical along their lengths. The central convex region 22 will have a generally spheroidal or ellipsoidal shape, as described below with reference to FIGS. 1F and 1G. While illustrated with a substantially symmetric geometry, the relative dimensions may of each region of the balloon vary widely with many specific examples illustrated below.

With reference to FIGS. 1F and 1G, a spheroidal geometry refers to a central convex region 22 which has an axial profile which is a chord 37 of a circle. The dimensions of the chord depend on the length of the central convex region 22 and the degree of curvature which is desired. Typically, the chord will have dimensions in the ranges of radius cr and angle x shown in Tables 1 and 2 hereinafter. Similarly, an ellipsoidal surface will be a chord 39 of an ellipse 38, shown in FIG. 1G. The dimensions of the chord 39 will be in the ranges of er1, er2, and angle y, also shown in Tables 1 and 2 below.

TABLE 1
REPRESENTATIVE DIMENSIONS
FOR 2 MM TO 5 MM BALLOONS

| | Exemplary | Specific | Preferred |
|---|---|---|---|
| Dimension | Range | range | Range |
| l₀ | 0 mm to 5 mm | 2 mm to 4 mm | 2.5 mm to 3.5 mm |
| l₁ | 4 mm to 38 mm | 5 mm to 28 mm | 6 mm to 28 mm |
| l₂ | 5 mm to 50 mm | 6 mm to 45 mm | 12 mm to 40 mm |
| l₃ | 0.1 mm to 10 mm | 0.5 mm to 8 mm | 1 mm to 6 mm |
| l₄ | 0.1 mm to 10 mm | 0.5 mm to 8 mm | 1 mm to 6 mm |
| l₅ | 0 mm to 2 mm | 0.1 mm to 1.5 mm | 0.5 mm to 1.5 mm |
| l₆ | 0.5 mm to 34 mm | 2 mm to 24 mm | 3 mm to 24 mm |
| α | 170° to 179.5° | 170° to 179° | 170° to 179° |
| Spheroidal | 100° to 170° | 125° to 170° | 150° to 170° |
| Ellipsoidal | | | |
| β | 0° to 9.5° | 0° to 9° | 0° to 8° |
| Spheroidal | 0° to ±25° | 0° to ±12° | 0° to ±2° |
| Ellipsoidal | | | |
| γ | 0.5° to 10° | 1° to 10° | 2° to 10° |
| Spheroidal | 10° to 80° | 10° to 60° | 10° to 30° |
| Ellipsoidal | | | |
| d₁ | 0.3 mm to 3 mm | 0.4 mm to 2 mm | 0.6 mm to 1.5 mm |
| d₂ | 2 mm to 5 mm | 2 mm to 4.5 mm | 2.5 mm to 4 mm |
| d₃ | 3 mm to 6 mm | 2 mm to 5.5 mm | 2.75 mm to 4.5 mm |
| rd | 1 mm to 2.5 mm | 1 mm to 2.5 mm | 1.4 mm to 2.25 mm |
| rc | 1 mm to 3.5 mm | 1 mm to 3 mm | 1.6 mm to 2.6 mm |
| rp | 1 mm to 2.5 mm | 1 mm to 2.5 mm | 1.4 mm to 2.25 mm |
| cr | 1 mm to 4000 mm | 40 mm to 300 mm | 60 mm to 200 mm |
| x | 2° to 50° | 3° to 40° | 2° to 10° |
| er1 | 2 mm to 15 mm | 3 mm to 10 mm | 4 mm to 8 mm |
| er2 | 0.1 mm to 3 mm | 0.2 mm to 1 mm | 0.25 mm to 0.5 mm |
| y | 2° to 50° | 3° to 40° | 2° to 10° |

TABLE 2
REPRESENTATIVE DIMENSIONS
FOR BALLOONS LARGER THAN 5 MM

| Dimension | Broad | Preferred | More preferred |
|---|---|---|---|
| mm or ° | Range | Range | Range |
| l₀ | 0 mm to 10 mm | 0 mm to 8 mm | 0 mm to 6 mm |
| l₁ | 4 mm to 150 mm | 10 mm to 120 mm | 20 mm to 100 mm |
| l₂ | 20 mm to 200 mm | 30 mm to 150 mm | 30 mm to 100 mm |
| l₃ | 3 mm to 50 mm | 4 mm to 25 mm | 5 mm to 25 mm |
| l₄ | 20 mm to 200 mm | 30 mm to 150 mm | 30 mm to 100 mm |
| l₅ | 0 mm to 5 mm | 0.5 mm to 2 mm | 0.5 mm to 1 mm |
| l₆ | 3 mm to 140 mm | 6 mm to 100 mm | 10 mm to 80 mm |
| α | 170° to 179.5° | 170° to 179° | 170° to 179° |
| Spheroidal | 100° to 170° | 125° to 170° | 150° to 170° |
| Ellipsoidal | | | |
| β | 0° to 9.5° | 0° to 9° | 0° to 8° |
| Spheroidal | 0° to ±25° | 0° to ±12° | 0° to ±2° |
| Ellipsoidal | | | |
| γ | 0.5° to 10° | 1° to 10° | 2° to 10° |
| Spheroidal | 10° to 80° | 10° to 60° | 10° to 30° |
| Ellipsoidal | | | |
| d₁ | 0.5 mm to 4 mm | 1.5 mm to 3 mm | 1.5 mm to 2 mm |
| d₂ | 4 mm to 14 mm | 5 mm to 12 mm | 5 mm to 12 mm |
| d₃ | 4.1 mm to 16 mm | 5.20 mm to 13 mm | 5.25 mm to 13 mm |
| rd | 2 mm to 7 mm | 2.25 mm to 6 mm | 2.25 mm to 6 mm |
| rc | 4.1 mm to 16 mm | 5.20 mm to 13 mm | 5.25 mm to 13 mm |
| rp | 4 mm to 14 mm | 5 mm to 12 mm | 5 mm to 12 mm |
| cr | 10 mm to 4000 mm | 40 mm to 300 mm | 60 mm to 200 mm |
| x | 2° to 50° | 3° to 40° | 2° to 10° |
| er1 | 2 mm to 15 mm | 3 mm to 10 mm | 4 mm to 8 mm |
| er2 | 0.1 mm to 3 mm | 0.2 mm to 1 mm | 0.25 mm to 0.50 mm |
| y | 2° to 50° | 3° to 40° | 2° to 10° |

In addition to the central convex region 22, proximal flanking region 24, and distal flanking region 26, the balloons 14 of the present invention will also have proximal and distal terminal or cone regions 34A and 34B. Although these regions are illustrated as cones in FIG. 1B and elsewhere in the present drawings, it will be appreciated that these terminal regions can't have any geometry of a type is known in the manufacture of generally cylindrical stent delivery catheter balloons. The proximal and distal terminal or cone regions 34A and 34B will generally not be involved directly in the balloon dilatation and/or stent expansion so that their particular dimensions and constructions are not a critical part of the present invention.

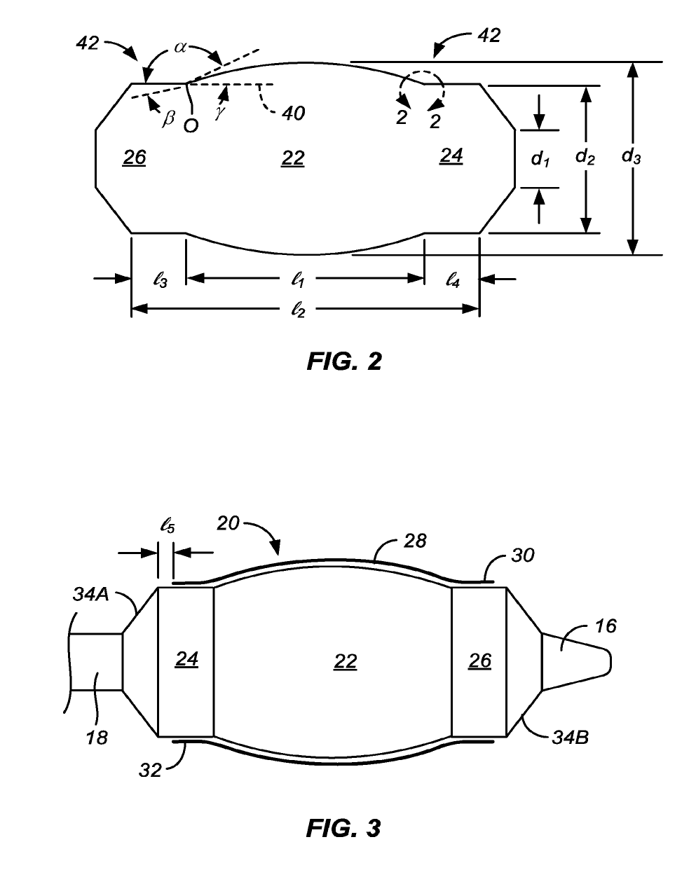

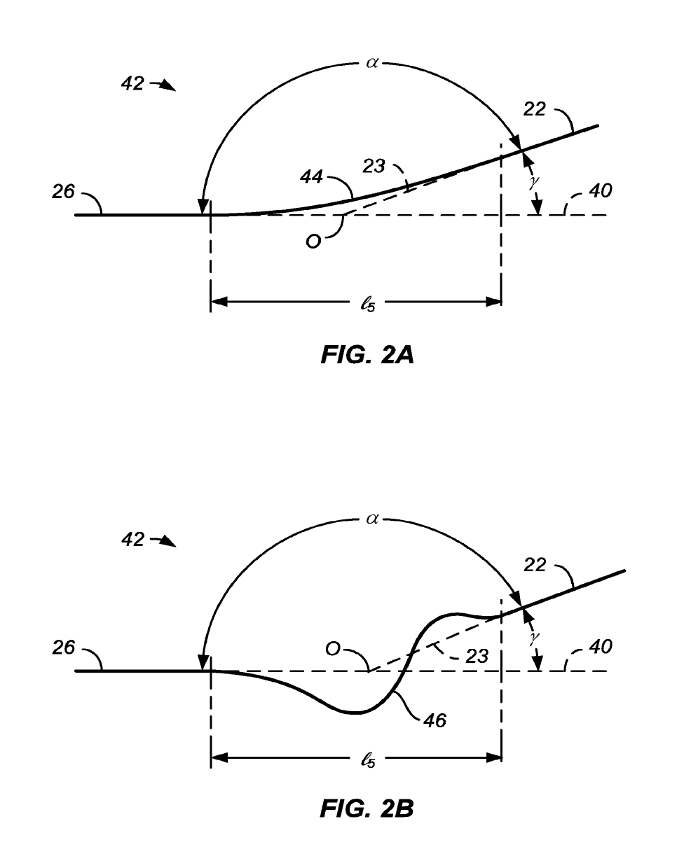

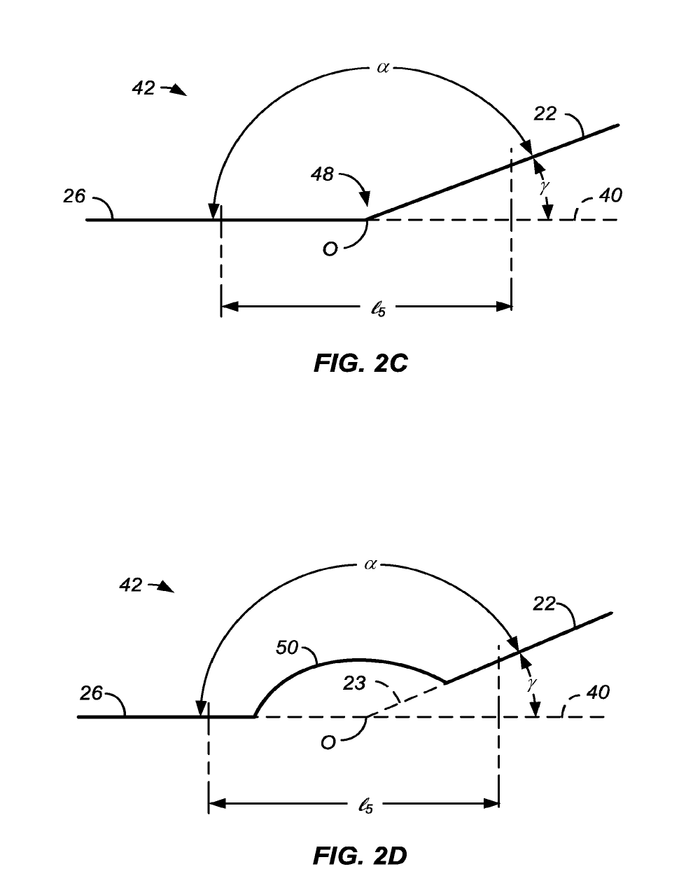

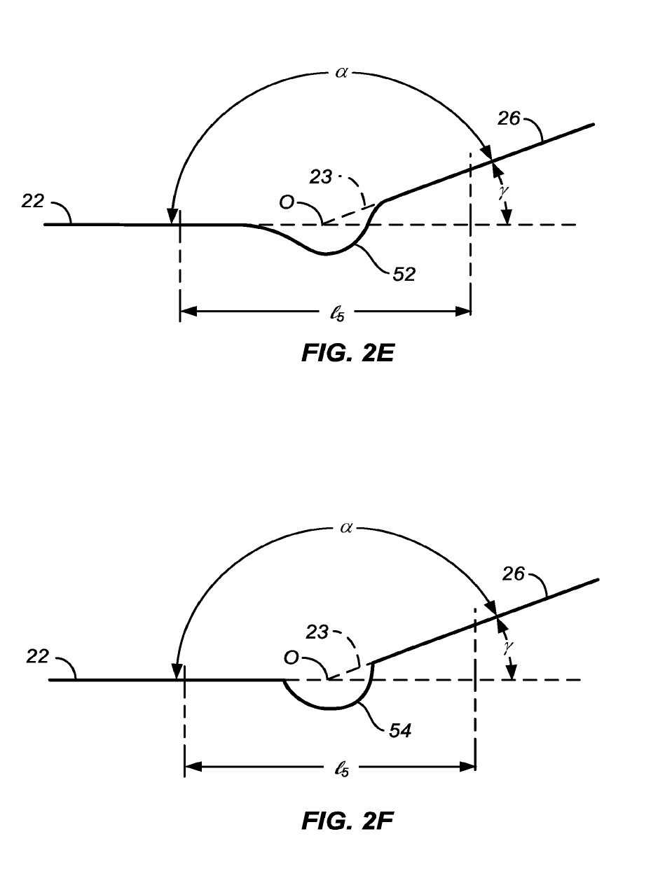

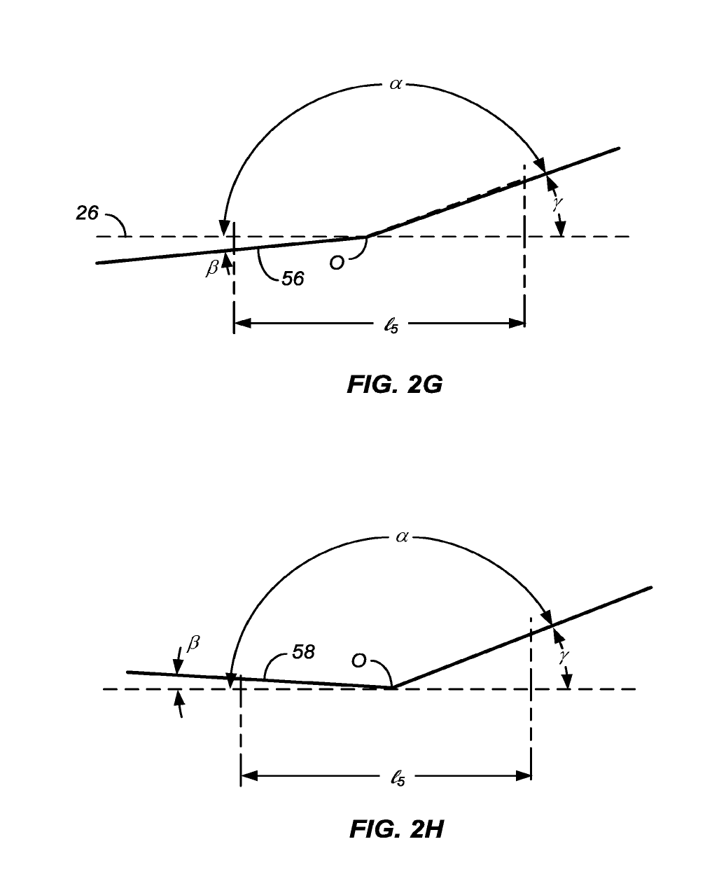

Referring now to FIG. 2, an important aspect of the present invention is the nature of a transition region 42 between the flanking regions 24 and 26 and the adjacent edges of the central convex region 22. As shown, at the transition between the distal flanking region 26 and the distal end of the central convex region 22, the distal edge of the central convex region will join the distal flanking region 26 at a transition angle a. The exemplary, specific, and preferred ranges for the value of the transition angle a are set forth in Tables 1 and 2 above. The angle of the distal edge of the central convex region 22 can also be measured relative to an axial line 40 which passes through the same origin O through which passes the junction point between the central convex region 22 and the distal flanking 26. This angle is noted in FIG. 2 as angle y. Finally, FIG. 2 also shows an angle 3 which represents the takeoff direction of the distal flanking region 26 from the origin point O. Usually 3 will be zero, i.e., the distal flanking region will be generally cylindrical with walls parallel to the axial line 40 (flat or substantially flat). In other cases, 3 may be a small positive angle, in which case the flanking region is tapered inwardly in the distal direction. Alternatively, the angle of 3 may be negative, in which case the flanking region flares outwardly in the distal direction. Of course, such inward tapering or outward flaring may be limited to a small portion of the length of the proximal and/or distal flanking region, and different details on such junctions are described below with reference to FIGS. 2A through 2H.

Other dimensional ranges for the exemplary balloon FIG. 2 are also set forth in Tables 1 and 2. In particular, d1 represents the outside diameter of the catheter shaft to be attached to the balloon. Dimension d2 represents the nominal diameter of the inflated proximal flanking region 24. Usually, but not necessarily, the diameter of the proximal and distal flanking regions will be the same. In most cases, even if different, the nominal diameters of the proximal and distal flanking regions will be within the ranges set forth in Tables 1 and 2. Dimension d3 represents the maximum inflated diameter of the central convex region 22 of the balloon. In other cases, the nominal diameter of the proximal flanking region is larger than the nominal diameter of the distal flanking region ranging from 0.1 mm larger to 0.5 mm larger, preferably 0.15 mm larger to 0.25 mm larger, however the transition angles remain within the desired ranges described in this application. The diameter in a preferred example of at least one of the flanking region(s) may generally correspond to a labeled or nominal balloon diameter.

Referring now to FIG. 3, the stent 20 will be placed over the balloon 14 so that, after balloon inflation, the stent will span the entire length of the central convex region 22 as well as at least a portion of each of the proximal flanking region 24 and the distal flanking region 26, as illustrated. Usually, the stent will span substantially the entire length of all three of these regions, with the setback 15 being within the range set forth in Tables 1 and 2 above. To achieve this setback, the stent is typically placed on the balloon 14 with a setback 10, as shown in FIG. 1, within the ranges shown in Tables 1 and 2. As can be seen in FIG. 3, even though the stent 20 is typically cylindrical in its initial configuration and would be cylindrical if expanded by a cylindrical delivery balloon, the stent assumes a contoured configuration with a domed central region 28 with two adjacent flat regions 30 and 32.

Referring now to FIGS. 2A to 2F, the transition region 42 may have a wide variety of configurations. As shown in FIG. 2A, the transition region may comprise a generally smooth curve 44 between a proximal end of distal flanking region 26 and a distal edge of the center convex region 22. The angle y will remain within the ranges of Tables 1 and 2 and will be measured between a tangential line 23 aligned with a distal edge of the central convex region 22. Tangential line 23 and an axial line 40 will meet at a hypothetical origin O which provides a point at which all angles a, f3, and y may be measured.

As shown in FIG. 2B, the proximal end of the distal flanking region 26 and distal edge of the central convex region 22 may be oriented at identical angles a and y from above the transition region itself may have quite a different sinusoidal or serpentine configuration 46. In FIG. 2C, a similar distal flanking region 26 and central convex region 22 are joined at a sharp or abrupt point 48. In FIG. 2D, the distal flanking region 26 and central convex region 22 are joined with a short convex segment 50. In FIG. 2E, the joining element is a short concave segment 52 with generally arcuate junctions with the distal flanking region 26 and center convex region 22. Finally, as shown in FIG. 2F a short concave segment 54 is joined to the distal flanking region 26 and the central convex region 22 by sharp or abrupt connection points.

While these transition regions 42 may have widely varying geometries, they will be present over very short lengths 15. As these lengths are so short, the different specific geometries have little impact on the expansion characteristics of the balloon and the ability of the balloons to inflate the stents that they are carrying with minimal stress and damage. In contrast, the transition angles α and γ should be kept within the ranges set forth in Tables 1 and 2 in order to avoid creating a large step or shearing element which would contact the inner surface of the stent when the balloon is expanded. It is these large steps in the stepped balloons of the prior art which are to be avoided in the present invention.

Turning now to FIGS. 2G and 2H, specific angles and dimensions of a converging flanking region 56 and a diverging flanking region 58 are shown. The specific values for angles α, β, and γ are shown in Tables 1 and 2 above.

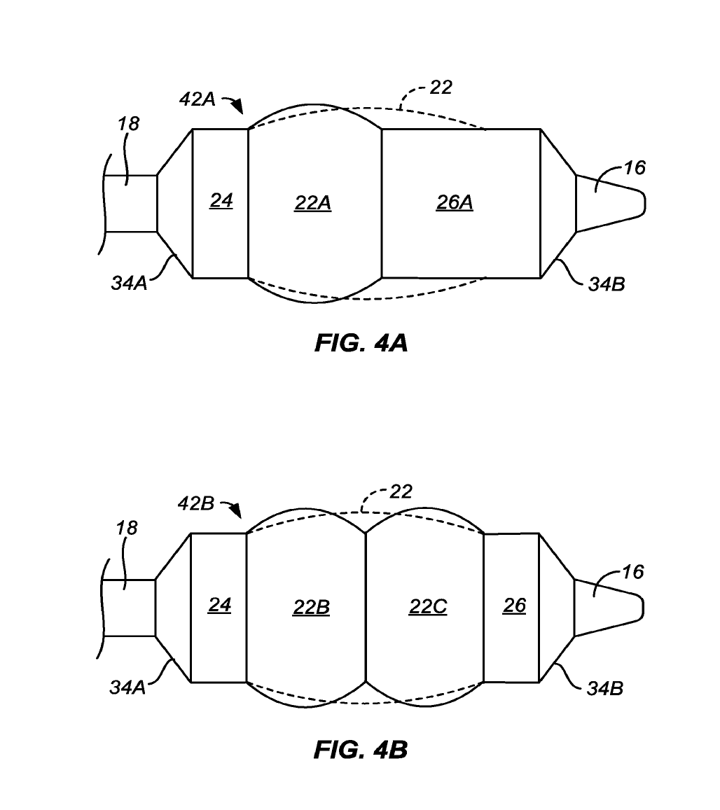

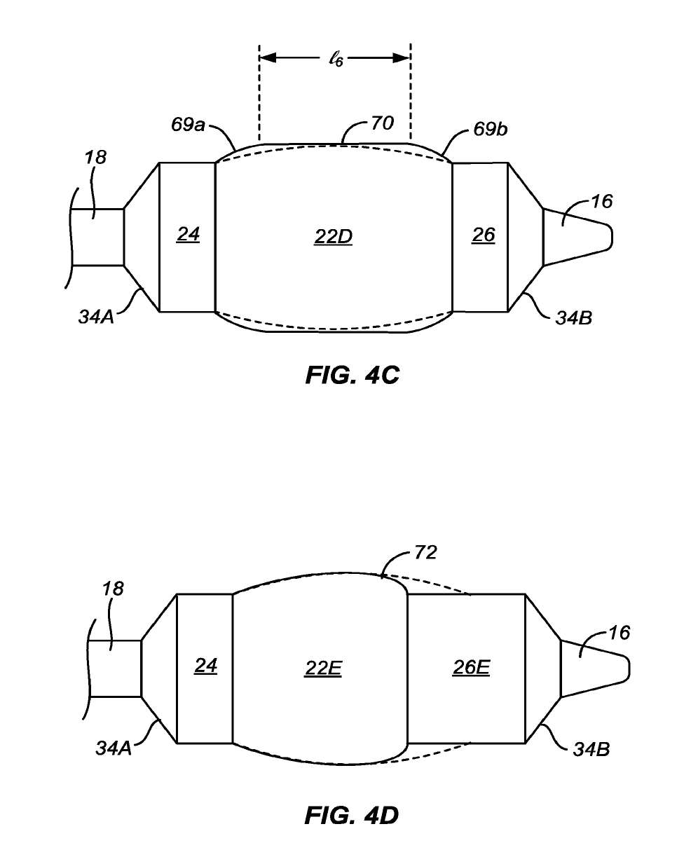

Referring now to FIGS. 4A through 4D, a variety of different central convex regions or balloons in accordance with the principles of the present invention will be described. In FIG. 4A, a central convex region 22A may be asymmetrically located between a proximal flanking region 24 and extended distal region 26A, for example. The geometry of the prior central convex region 22 is shown in broken line for comparison. In FIG. 4B, the central convex region may comprise a pair of shorter central convex regions 22B and 22C. While the transition regions 42A in FIG. 4A and 42B in FIG. 4B appear to be larger than those described in the previous embodiments, will be appreciated that the angles themselves will still be within the ranges set forth in Tables 1 and 2 above. In FIG. 4C, a central convex region 22D may have proximal and distal contoured or “semi-domed” regions 69a and 69b, with a generally flat region 70 therebetween. The flat region may have a length 16 within the rages set forth above in Tables 1 and 2. Alternatively, the central flat section 70 can have a variable diameter forming a taper from distal to the proximal ends of the flat section as shown in FIG. 4E. The angles and dimensions of the semi-domed transition regions will be selected as with all embodiments herein to avoid the application of excessive shearing and other forces to the stent when the balloon is expanded. In FIG. 4D, a central convex region 22E may be asymmetrically shaped with a lower transition angle on the proximal end and a more curved region 72 on the distal end. The central region 22E is shown to be asymmetrically knotted within the balloon as a whole, but it will be appreciated that it can also be centrally located on the balloon.

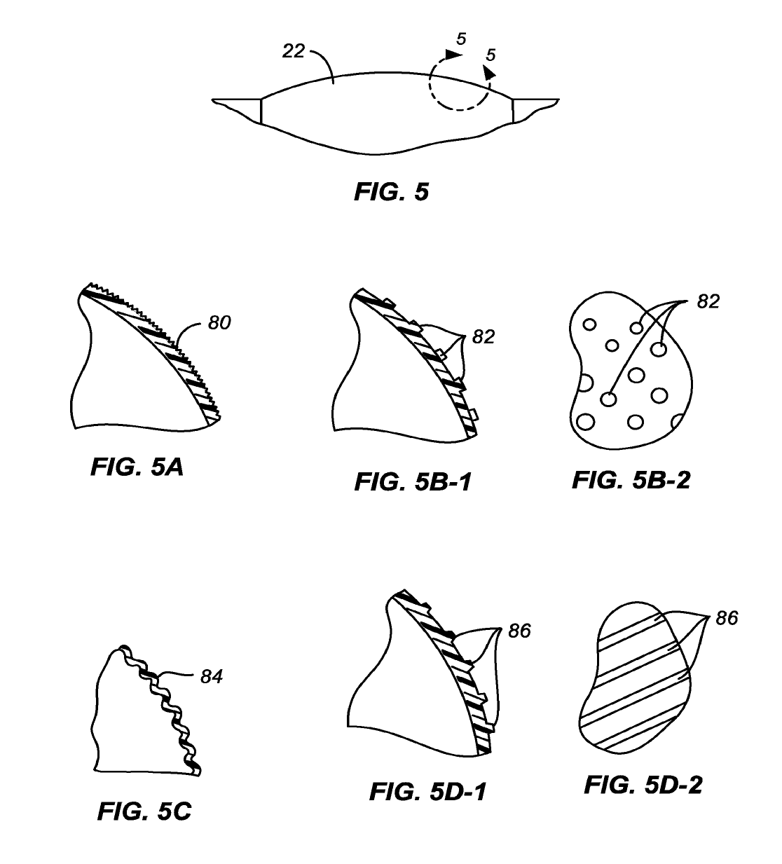

Referring now to FIG. 5, a surface of the central region 22 may be smooth and generally have the characteristics of known stent delivery catheter balloons. Alternatively, as shown in FIGS. 5A through 5D-2, the surface may be modified to have various features to enhance the interface between the balloon and the stent as the balloon is expanded. For example, as shown in FIG. 5A, the balloon may have a sawtooth or serrated surface 80. Referring now to FIGS. 5B-1 and 5B-2, the balloon surface may have a series of bumps or nubs 82 formed over its surface in order to enhance the interface with the stent being delivered. Similarly, as shown in FIG. 5C, the balloon surface may be corrugated. Additionally, as shown in FIGS. 5D-1 and 5D-2, the balloon surface may have a series of ribs formed thereover. As specifically shown on the central convex region 22 of the inflation balloon 14, will be appreciated that the surface modifications may be present on the flanking regions as well and may be formed over only a portion of any of these regions.

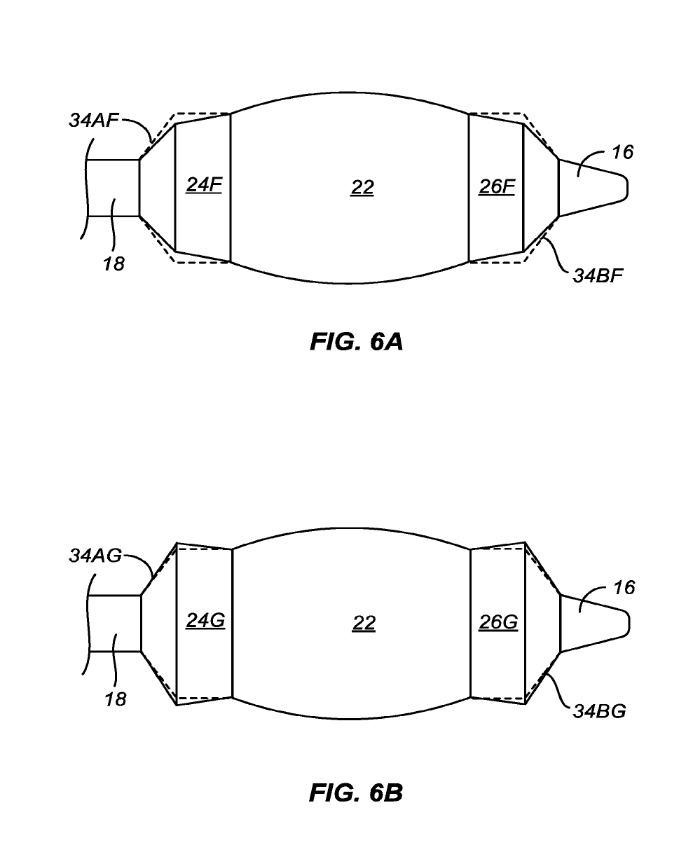

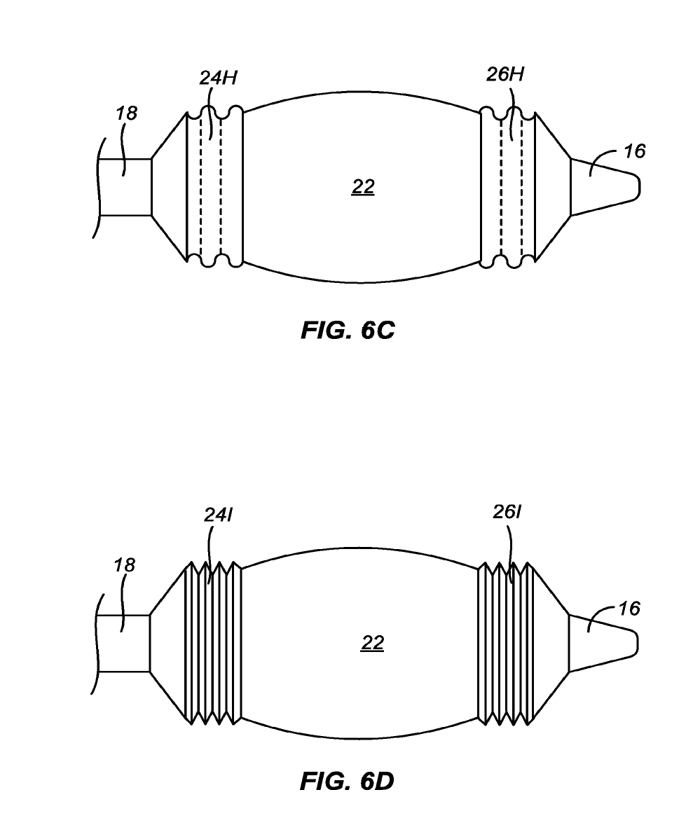

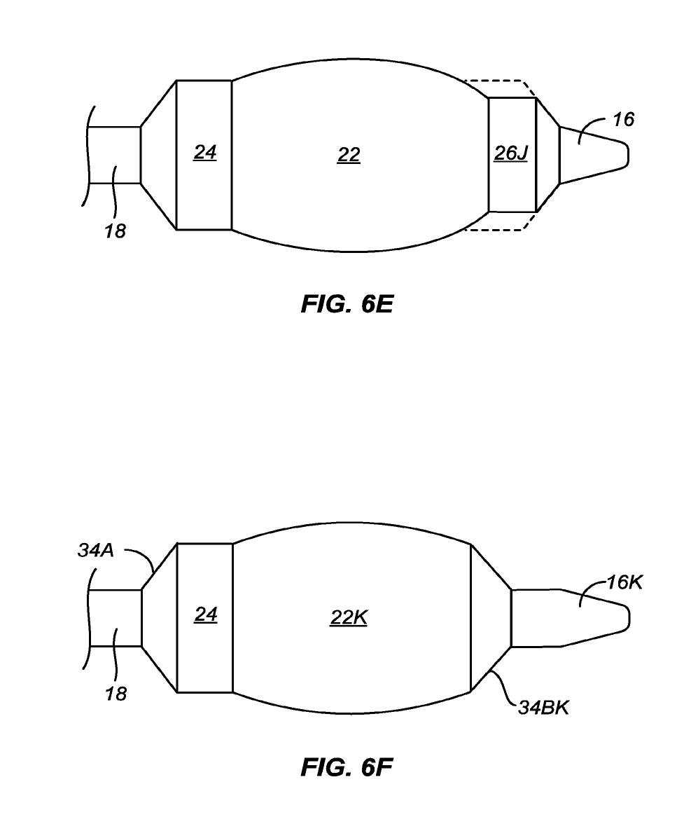

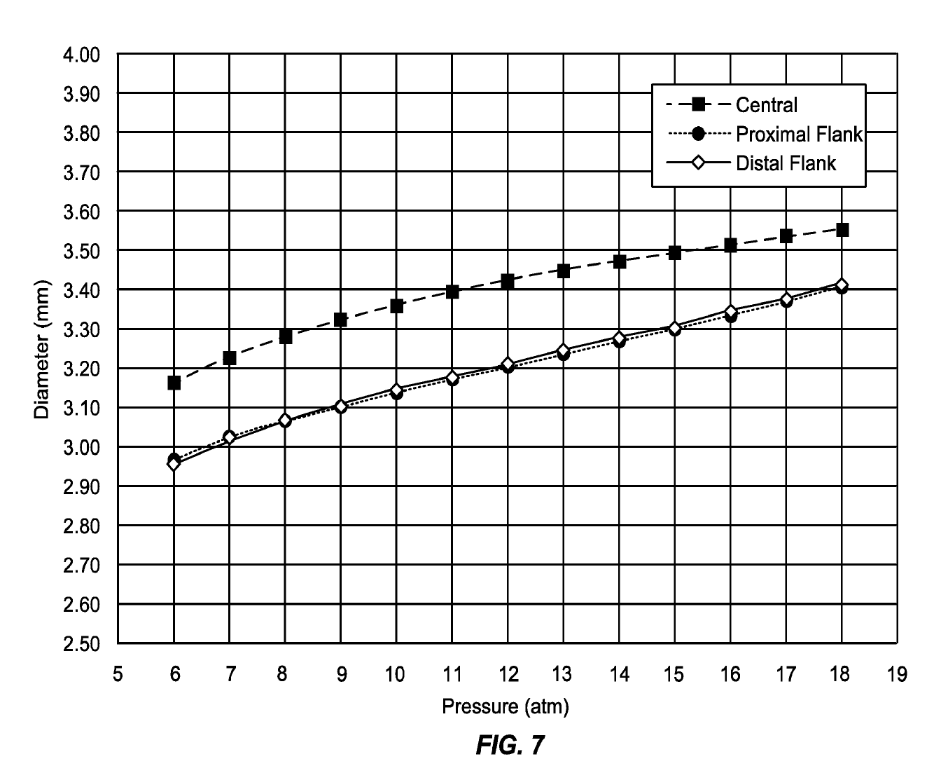

Referring now to FIG. 6A through 6F, the proximal and distal flanking regions 24 and 26 may have a variety of configurations in addition to the cylindrical configurations shown previously. In FIG. 6A, the proximal and distal flanking regions 24F and 26F are shown to be tapered inwardly in the directions away from the central convex region 22. The true cylindrical shapes are shown in broken line for comparison. In FIG. 6B, proximal and distal flanking regions 24G and 26G are shown with surfaces that taper radially outwardly in the directions away from the central convex region 22. Again, the true cylindrical geometries are shown in broken line. In FIG. 6C, proximal and distal flanking regions 24H and 26H are shown with corrugated configurations, while in FIG. 6D, proximal and distal flanking regions 24I and 26I are shown with pleated surfaces. As shown in FIG. 6E, the dimensions of the proximal flanking region 24 and distal flanking region 26J need not be identical or can be different, and as shown in FIG. 6F, in some examples or embodiments, only a single flanking region, such as proximal flanking region 24, need be provided and the other side of the central region 22K can have a conventional conical or other terminal region 34BK. The flanking regions can have more than one shape or portion of shapes described previously.

The following paragraphs provide specific examples of preparing balloons and stents in accordance with the principles of the present invention.

EXAMPLE 1: An inflatable 3.0 mm diameter by 18 mm length balloon (labeled) attached to a distal end of a balloon catheter was inserted into a mold having with proximal and distal sections flanking a central convex section. The balloon was inflated within the cavity while simultaneously applying heat and pressure to form a convex central region in the balloon. The balloon was deflated, cooled, and removed from the mold. The balloon wall was measured to have a thickness of 20 microns which was substantially the same in the convex region and the flanking regions. The balloon was inflated to its nominal 3.0 mm diameter. The proximal and distal flanking regions diameters were approximately 3.0 mm when inflated to the nominal inflation pressure. The maximum diameter of the central convex region was measured at approximately the center of the balloon working length and to be approximately 3.25 mm. The balloon was inflated to RBP where the flanking regions maximum diameter was measured to be approximately 3.3 mm while the maximum diameter of the convex central region was measured to be approximately 3.5 mm. The proximal flanking region at nominal inflation pressure was substantially flat and had a length of approximately 3 mm. The distal flanking region at nominal inflation pressure was substantially flat and had a length of approximately 3 mm. The length from the transition angle to the maximum diameter of the convex central lumen (along the catheter length) was measure to be approximately 6 mm. The total convex central region length was approximately 12 mm, and the total flanking regions length of both proximal and distal was 6 mm, providing a total working length of 18 mm for the inflatable balloon. The transition angle between the convex central region and the adjacent flanking regions was measured to be the same for both distal and proximal flanking regions and was measured to be 176°. The delivery system was labeled as 3.0 mm diameter by 18 mm. Typical balloon dimensions at nominal inflation pressures are as in Table 3 below for both a 18 mm×3 mm balloon and a 28 mm×3.5 mm balloon:

TABLE 3

| | Balloon | |
|---|---|---|
| | Working Length | |
| Typical balloon Dimension | 18 mm | 28 mm |
| Balloon nominal diameter | 3.0 | 3.5 |
| (d₂) (mm) | | |
| Balloon Taper length (mm) | 3.0 | 3.5 |
| Balloon Working | | |
| length (f₂) (mm) | 18 | 28 |
| Contour length | 6 | 6 |
| (½ L1) (mm) | | |
| Flank length | 3.00 | 3.00 |
| ( 13/4) ( mm) | | |
| Stent offset from | 0-0.5 | 0-0.5 |
| flank ends (15) (mm) | | |
| Flat section on central | 0 | 10 |
| convex (16) (mm) | | |
| Transition angle α (°) | 176 | 176 |
| β (°) | ~0 | ~0 |
| γ (°) | 3.69 | 3.69 |
| Balloon shaft junction | 0.7-1.0 | 0.8-1.0 |
| diameter (d₁) (mm) | | |
| Flange diameter | 3 | 3 |
| (rd and rp) (mm) | | |
| Central section | 3.25 | 3.75 |
| diamter (d₃) (mm) | | |
| cr (mm) | 72 | 72 |
| α (°) | 10.00 | 10.00 |

EXAMPLE 2: An inflatable 3.0 mm diameter by 18 mm length balloon (labeled) attached to a distal end of a balloon catheter is inserted into a mold having a central convex shape and one flanking regions distal to the central convex region. The balloon is inflated within the cavity while simultaneously applying heat and pressure to form a convex central region in the balloon. The balloon is deflated, cooled, and removed from the mold. The balloon wall is measured to have a thickness of 20 microns which is substantially the same in the convex region and the single flanking region. The balloon is inflated to the nominal inflation pressure. The distal flanking region at nominal inflation pressure is measured to have a 3.0 mm diameter. The diameter of the convex central region adjacent to the proximal conical end is measured to be 3.05 mm. The maximum diameter of the central convex region is measured to be 3.25 mm. The balloon is then inflated to RBP, and the distal flanking region diameter is measured to be approximately 3.3, the proximal end of the convex central region adjacent the conical end is measured to be 3.4 mm, and the maximum diameter of the convex central region is measured to be 3.6 mm. The distal flanking region at nominal inflation pressure is substantially flat and had a length of approximately 3 mm. The length from the transition angle to the maximum diameter of the convex central lumen (along the catheter length) is measured to be 7.5 mm. The total convex central region length is measured to be 15 mm, and the length of the distal flanking region is about 3 mm, providing a total working length of about 18 mm for the inflatable balloon. The transition angle between the convex central region and the adjacent distal flanking regions is measured to be 176°.

EXAMPLE 3: An inflatable balloon catheter labeled 3.0 mm diameter by 18 mm length having the balloon attached to the proximal and distal ends of a catheter or catheter body. The balloon is inserted into a mold having a central convex shape in the longitudinal direction, and two flanking regions proximal and distal to the central convex region. The balloon is inflated while simultaneously heated and pressurized to form the convex central region. The balloon is deflated, cooled, and removed from the mold. The balloon thickness of 20 microns is measured to be substantially the same in the convex region and the flanking regions. The balloon is inflated to the nominal 3.0 mm diameter. The proximal and distal flanking regions diameters at nominal were approximately 3.0 mm. The maximum diameter of the central convex region is measured to be approximately 3.25 mm. The balloon is inflated to RBP, at RBP, the flanking regions diameters were measured to be approximately 3.3 mm while the maximum diameter of the convex central region is measured to be approximately 3.5 mm. The proximal flanking region at nominal is substantially flat and had a length of approximately 3 mm. The distal flanking region at nominal is substantially flat and had a length of approximately 3 mm. The length from the proximal transition angle to the maximum diameter of the convex central lumen (along the catheter length) is measured to be approximately 4 mm, while the length from the distal transition angle to the maximum diameter of the convex central lumen is measured at approximately 8 mm. The total convex central region length is 12 mm, and the total flanking regions length is 6 mm, providing a total working length of 18 mm for the inflatable balloon. The transition angle between the convex central region and the adjacent flanking regions is measured to be 176° distal and proximal flanking regions and is measured to be 176°. The delivery system is labeled 3.0 mm by 18 mm, the diameter of the proximal and distal flanking regions (at nominal), and the working length of the inflatable balloon.

EXAMPLE 4: An inflatable balloon catheter labeled 3.0 mm diameter or 2.85 mm diameter, by 18 mm length having the balloon attached to the proximal and distal ends of a catheter or catheter body. The balloon is inserted into a mold having a central convex shape in the longitudinal direction, and two flanking regions proximal and distal to the central convex region. The balloon is inflated while simultaneously heated and pressurized to form the convex central region and larger proximal flanking region. The balloon is deflated, cooled, and removed from the mold. The balloon thickness 20 microns is measured to be substantially the same in the convex region and the flanking regions. The balloon is inflated to the nominal diameter. The proximal and distal flanking regions diameters at nominal were approximately 3.0 mm and 2.85 mm respectively. The maximum diameter of the central convex region is measured at approximately the center of the balloon working length and is measured to be approximately 3.3 mm at nominal. The balloon is inflated to RBP, at RBP, the flanking regions diameters were measured to be approximately 3.3 mm for proximal, 3.15 mm for the distal while the maximum diameter of the convex central region is measured to be approximately 3.55 mm. The proximal flanking region at nominal is substantially flat and had a length of approximately 3 mm. The distal flanking region at nominal is substantially flat and had a length of approximately 3 mm. The length from the transition angle to the maximum diameter of the convex central lumen (along the catheter length) is measured to be approximately 6 mm. The total convex central region length is 12 mm, and the total flanking regions length is 6 mm, providing a total working length of 18 mm for the inflatable balloon. The transition angle between the convex central region and the adjacent proximal flanking regions is measured to be 176° while the distal transition angle is measured to be 176°. The delivery system can be labeled as 2.85 mm by 18 mm (the diameter of the distal flanking region at nominal pressure), 3.0 mm by 18 mm (the diameter of the proximal flanking region at nominal pressure), 2.925 mm by 18 mm (the mean between proximal flanking region and distal flanking region diameters), or 3.0 mm×2.85 mm by 18 mm (the diameters of both the proximal and distal flanking regions respectively). The 18 mm is the balloon working length. Additional labeling for the maximum diameter of the central convex region for at least the nominal and RBP pressures.

EXAMPLE 5: An inflatable 3.0 mm diameter by 28 mm length balloon (labeled) attached to a distal end of a balloon catheter is inserted into a mold having with proximal and distal sections flanking a central convex section. The central convex section has a substantially flat section at its center. The balloon is inflated within the cavity while simultaneously applying heat and pressure to form a convex central region in the balloon. The balloon is deflated, cooled, and removed from the mold. The balloon wall is measured to have a thickness of 20 microns which is substantially the same in the convex region and the flanking regions. The balloon is inflated to its nominal 3.0 mm diameter. The proximal and distal flanking regions diameters were measured to be 3.0 mm when inflated to the nominal inflation pressure. The maximum diameter of the central convex region is measured to be 3.35 mm at a substantially flat center segment of the balloon produced by the flat section of the mold cavity. The balloon is inflated to RBP, and the flanking regions diameters were measured to be 3.3 mm while the diameter of the flat center segment of the convex central region is measured to be 3.65 mm. The proximal flanking region at nominal inflation pressure is substantially flat and had a measured length of 3 mm. The distal flanking region at nominal inflation pressure is substantially flat and had a measured length of 3 mm. The length from the transition region to the maximum diameter of the convex central lumen (along the catheter length) is measured to be approximately 6 mm from both the proximal and distal adjacent regions. The length of the flat segment in the central convex region is measured to be 10 mm. The total substantially convex central region including the flat segment length is measured to be 22 mm, and the total length of both flanking regions is 6 mm, providing a total working length of 28 mm for the inflatable balloon. The transition angle between the convex central region and the adjacent flanking regions is measured to be 176° for both the distal and the proximal flanking regions.

EXAMPLE 6: A substantially non-degradable stent is patterned from a tube or formed from a wire. The stents comprise a plurality of rings including structural elements, e.g. struts joined by crowns, and each ring is connected to an adjacent ring in at least one location. At least some structural elements or at least some rings have cross sectional area ranging from 2500 mm² to 5500 mm². The stent (18 mm or 28 mm long) is crimped onto a suitable length delivery system of examples 1 through 5, where the stent substantially spans the entire working length of the balloon (±1 mm). The stent is deployed to an expanded larger configuration from the crimped configuration. The central region of the stent adjacent to the convex central region of the balloon is expanded to a larger diameter than at least one of the adjacent proximal and/or distal regions. The stent after balloon deflation inward recoils by a magnitude of 0.05 mm to 0.175 mm. The stent diameter in the maximum central region is substantially maintained to be larger than at least one adjacent proximal and/or distal regions. The stent is deployed in air, in water, in water at 37C, and/or under other physiological conditions.

EXAMPLE 7: A substantially non-degradable stent is patterned from a tube or formed from a wire. The stents comprise a plurality of rings including structural elements, e.g. struts joined by crowns, and each ring is connected to an adjacent ring in at least one location. At least some structural elements or at least some rings have cross sectional area ranging from 2500 μm² to 5500 μm². The stent (18 mm or 28 mm long) is crimped onto a suitable length delivery system of examples 1 through 5, where the stent substantially spans the entire working length of the balloon (±1 mm). The stent is deployed in a mammalian diseased artery to an expanded larger configuration from the crimped configuration. The central region of the stent adjacent to the convex central region of the balloon is expanded at least is some portion of the central region to a larger diameter than at least one of the adjacent proximal and/or distal regions. The stent after balloon deflation exhibit inward recoil equal to or larger than the maximum expanded diameter of the central region. The % diameter stenosis post implant is 0% to 15%, an optimal or acceptable result, while the % stenosis would have been about 20% or greater if the stent is deployed using conventional balloon.

EXAMPLE 8: A degradable stent is formed from a degradable PLLA-based polymeric material. The stent is patterned from polymer filaments or from a polymer tube. The patterned stent comprises structural elements, e.g. struts joined by crowns forming a plurality of rings, where each ring is connected to an adjacent ring in at least one location. The stent (18 mm or 28 mm in length) is crimped onto a suitable length delivery system of Examples 1 through 5, where the stent substantially spans the working length of the balloon. The stent is deployed to an expanded larger configuration from the crimped configuration. The central region of the stent adjacent to the convex central region of the balloon is expanded to a larger diameter than at least one of the adjacent proximal and/or flanking distal regions. The stent after balloon deflation recoils inwardly from 2% to 10% of the expanded diameter. At least a portion of the stent in the central region has a diameter which is substantially maintained to be larger than at least one adjacent proximal and/or distal flanking regions. The stent is deployed in water at 37° C., and/or under other physiological conditions. The stent is expandable from 0.5 mm to 1 mm above nominal diameter without fracture.

EXAMPLE 9: A degradable stent is formed from a degradable PLLA-based polymeric material. The stent is patterned from polymer filaments or from a polymer tube. The patterned stent comprises structural elements, e.g. struts joined by crowns forming a plurality of rings, where each ring is connected to an adjacent ring in at least one location. The weight of the polymeric degradable material is 0.75 mg/mm of stent length, i.e. a stent weight of 13.5 mg for 18 mm stent, or 21 mg for a 28 mm stent. The degradation period for the material ranges from 3 months to 2 years. The stent (18 mm or 28 mm in length) is crimped onto a suitable length delivery system of Examples 1 through 5, where the stent substantially spans the working length of the balloon. The stent is deployed to an expanded larger configuration from the crimped configuration. The central region of the stent adjacent to the convex central region of the balloon is expanded to a larger diameter than at least one of the adjacent proximal and/or distal regions. The stent after balloon deflation inward recoils from 2% to 10% of the expanded diameter, at least a portion of the stent in the central region has a diameter which is larger than at least one adjacent proximal and/or distal region. The stent is deployed in water at 37° C., and/or under other physiological conditions. The stent is expandable without fracture, e.g. expandable from 0.5 mm to 1 mm above nominal diameter without fracture. Expansion of the stent with a balloon having a convex shape with a transition angle in the ranges set forth above and maximum diameter in the convex region of the stent larger than at least one adjacent proximal and/or distal regions allows the stent to have sufficient strength to support a body lumen.

EXAMPLE 10: A degradable stent is formed from a degradable PLLA-based polymeric material. The stent is patterned from polymer filaments or from a polymer tube. The patterned stent comprises structural elements, e.g. struts joined by crowns forming a plurality of rings, where each ring is connected to an adjacent ring in at least one location. The degradation period for the material ranges from 3 months to 2 years. At least some of the structural elements have cross sectional area ranging from 14000 μm² to 25000 μm². In another example, at least some of the structural elements rings have cross sectional area ranging from 14000 μm² to 25000 μm². In another example substantially all of the structural elements have cross sectional area ranging from 14000 μm² to 25000 μm². The stent (18 mm or 28 mm in length) is crimped onto a suitable length delivery system of examples 1 through 5, where the stent substantially spans the working length of the balloon. The stent is deployed to an expanded larger configuration from the crimped configuration. The central region of the stent adjacent to the convex central region of the balloon is expanded to a larger diameter than at least one of the adjacent proximal and/or distal regions. The stent after balloon deflation inward recoils from 2% to 10% of the expanded diameter, at least a portion of the stent in the central region has a diameter which is larger than at least one adjacent proximal and/or distal regions. The stent is deployed in water at 37° C., and/or under other physiological conditions. The stent is expandable without fracture, or expandable from 0.5 mm to 1 mm above nominal diameter without fracture. Expansion of the stent with a balloon having a convex shape with a transition angle in the ranges set forth above and maximum diameter in the convex region of the stent larger than at least one adjacent proximal and/or distal regions allows the stent to have sufficient strength to support a body lumen.

EXAMPLE 11: A degradable stent is formed from a degradable material. The stent is patterned from filaments or from a tube. The patterned stent comprises structural elements, e.g. struts joined by crowns forming a plurality of rings, where each ring is connected to an adjacent ring in at least one location. The degradation period for the material ranges from 3 months to 2 years. The stent has a 10% flat plate compression ranging from 0.15N/ to 0.4N (for 3.0 mm stent by 14 mm length). The stent (18 mm or 28 mm in length) is crimped onto a suitable length delivery system of examples 1 through 5, where the stent substantially spans the working length of the balloon. The stent is deployed to an expanded larger configuration from the crimped configuration. The central region of the stent adjacent to the convex central region of the balloon is expanded to a larger diameter than at least one of the adjacent proximal and/or distal regions. The stent after balloon deflation inward recoils from 2% to 10% of the expanded diameter, at least a portion of the stent in the central region has a diameter which is larger than at least one adjacent proximal and/or distal regions. The stent is deployed in water at 37C, and/or under other physiological conditions. The stent is expandable without fracture, or expandable from 0.5 mm to 1 mm above nominal diameter without fracture. Expansion of the stent with a balloon having a convex shape with a transition angle in the ranges set forth above and maximum diameter in the convex region of the stent larger than at least one adjacent proximal and/or distal regions allows the stent to have sufficient strength to support a body lumen.

EXAMPLE 12: A degradable stent is formed from a degradable material. The stent is patterned from filaments or from a tube. The patterned stent comprises structural elements, e.g. struts joined by crowns forming a plurality of rings, where each ring is connected to an adjacent ring in at least one location. The degradation period for the material ranges from 3 months to 2 years. The cross-sections of at least some of the stent structural elements have an abluminal convex (or dome) shape across the width of said structural element. The stent (18 mm or 28 mm in length) is crimped onto a suitable length delivery system of examples 1 through 5, where the stent substantially spans the working length of the balloon. The stent is deployed to an expanded larger configuration from the crimped configuration. The central region of the stent adjacent to the convex central region of the balloon is expanded to a larger diameter than at least one of the adjacent proximal and/or distal regions. The stent after balloon deflation inward recoils from 2% to 10% of the expanded diameter, at least a portion of the stent in the central region has a diameter which is larger than at least one adjacent proximal and/or distal region. The stent is deployed in water at 37° C., and/or under other physiological conditions. The stent is expandable without fracture, or expandable from 0.5 mm to 1 mm above nominal diameter without fracture. Expansion of the stent with a balloon having a convex shape with a transition angle in the ranges set forth above and maximum diameter in the convex region of the stent larger than at least one adjacent proximal and/or distal regions allows the stent to have sufficient strength to support a body lumen. The inflatable balloon having a convex central region embeds the structural elements having a substantially convex cross sectional shape when the stent is expanded from the crimped configuration to an expanded larger configuration, which improves blood flow dynamics, and/or reduce flow shear stresses.

Example 13: A stent comprising structural elements, the stent is formed from a wire and patterned, or formed from a tube and patterned. The structural elements comprise a plurality of rings, each ring comprises struts joined by crowns, and each ring is connected to an adjacent ring in at least one location. The stent (14 mm, 18 mm or 28 mm in length for example) is crimped onto a suitable length delivery system of examples 1 through 5, where the stent substantially spans the entire working length of the balloon. The stent is deployed in a mammalian diseased artery to an expanded larger configuration from the crimped configuration. The central region of the stent adjacent to the convex central region of the balloon is expanded at least is some portion of the central region to a larger diameter than at least one of the adjacent proximal and/or distal regions. Stent dimensions at nominal pressure and at rated burst pressure were as follows:

| | Stent Flank Distal | | | Central convex region | | Stent Flank Proximal | | |
|---|---|---|---|---|---|---|---|---|
| | OD | Length | Transition | OD | Length | OD | Length | Transition |
| Pressure | (mm) | (mm) | Angle ° | (mm) | (mm) | (mm) | (mm) | Angle ° |
| Nominal 7 | 3.26 | 3.63 | 178.90 | 3.47 | 7.31 | 3.25 | 2.50 | 178.61 |
| RBP 16 | 3.54 | 3.68 | 176.99 | 3.77 | 7.75 | 3.52 | 3.20 | 178.63 |

Although certain embodiments or examples of the disclosure have been described in detail, variations and modifications will be apparent to those skilled in the art, including embodiments or examples that may not provide all the features and benefits described herein. It will be understood by those skilled in the art that the present disclosure extends beyond the specifically disclosed embodiments or examples to other alternative or additional examples or embodiments and/or uses and obvious modifications and equivalents thereof. In addition, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments and examples may be made and still fall within the scope of the present disclosure. Accordingly, it should be understood that various features and aspects of the disclosed embodiments can be combined with or substituted for one another in order to form varying modes or examples of the present disclosure. Thus, it is intended that the scope of the present disclosure herein disclosed should not be limited by the particular disclosed embodiments or examples described above. For all of the embodiments and examples described above, the steps of any methods for example need not be performed sequentially.

1. A stent delivery catheter comprising
a catheter body having a proximal end, a distal end, and a longitudinal axis; and
an inflatable balloon at the distal end of the catheter body and having a central region, a proximal flanking region, and a distal flanking region;
wherein the central region has a convex shape relative to the flanking regions along the longitudinal axis and joins each flanking region at a transition angle α in the range from 160° to 179° when inflated.

2. A stent delivery catheter as in claim 1, wherein the central region comprises a spheroidal or ellipsoidal surface when the balloon is inflated.

3. A stent delivery catheter as in claim 2, wherein the spheroidal or ellipsoidal surface is uniformly curved between the proximal and distal flanking regions.

4. A stent delivery catheter as in claim 2, wherein the spheroidal or ellipsoidal surface has a greater curvature near its proximal and distal regions where the central region of the balloon meets the flanking regions.

5. A stent delivery catheter as in claim 1, wherein the convex central region comprises a proximal spheroidal or ellipsoidal surface region and a distal spheroidal or ellipsoidal surface region when the balloon is inflated, wherein the proximal and distal surface regions are joined by a flatter region therebetween.

6. A stent delivery catheter as in claim 1, wherein a surface of the convex central region is smooth when inflated.

7. A stent delivery catheter as in claim 1, wherein a surface of the convex central region is textured when inflated.

8. A stent delivery catheter as in claim 7, wherein a surface texture of the central convex region comprises features selected from the group consisting of corrugations, bumps, saw tooth elements, and ribs.

9. A stent delivery catheter as in claim 1, wherein the flanking regions are generally cylindrical.

10. A stent delivery catheter as in claim 1, wherein the flanking regions taper in diameter in a direction away from the central region, wherein a taper angle β of the flanking regions is less than a junction angle γ of the central convex region.

11. A stent delivery catheter as in claim 1, wherein the inflatable balloon is formed at least in part from a non-compliant material.

12. A stent delivery catheter as in claim 11, wherein the non-compliant material is selected from the group, consisting of polyethyleneterphthalate, polyamideimide copolymer, polyetherimide, polyetherketone, polyetheretherketone, polybutyleneterphthalate, polycarbonate, polyacetate, polyphthalamide, polycrylonitrile, polyarylene, polybutadiene, polyether, polyetherketones, polyimide, polyphenylenesulfide, polyphosphazenes, polyphosphonates, polysulfone, polycarbonate/polysulfone alloy, polysulfides, polsulfide, polythiophene, polyacetylene polycarbonates, polyphenylene ether, polyetherketones, polyimide, polyphenylene, Polycarbonate/polybutylene terephthalate alloy, ABS/PC blend, carbon reinforced composites, aramid fiber reinforced composites, poly [(R)-3-hydroxybutyrate-co-8%-(R)-3-hydroxyvalerate](P (3HB-co-8%-3HV)fibers composites, liquidcrystal fibers composites.

13. A stent delivery system catheter as in claim 1, wherein the inflatable balloon is formed at least in part from a semi-compliant material.

14. A stent delivery catheter as in claim 13, wherein the semi-compliant material is elected from the group consisting of polyamide (nylon 12, nylon 11, nylon 6-12, nylon 6-11, nylon 6-6, nylon 6,), nylon blends, nylon copolymers, polyetheramide copolymer, polyurethane, polyesterpolyurethane, poycarbonatepolyurethane, polyetherpolyurethane, polyolefinpolyamide, polyacrylonitrile, polytrimethyleneterephthalate, polyacrylonitrilebutadienestyrene, polyphenylsufone, polyphthalamide, polyaryletherketone, polyethersulfone, polybutyleneadipate, polyacetate, polyacrylate, ABS/Nylon blends, polycrylonitrile, polyanhydride, polyarylene.

15. A stent delivery catheter as in claim 1, wherein the central region and distal and proximal flanking regions have substantially the same compliance.

16. A stent delivery catheter as in claim 1, wherein the inflatable balloon has a single substantially uniform wall thickness.

17. A stent delivery catheter as in claim 1, wherein the inflatable balloon has a non-uniform wall thickness.

18. A stent delivery catheter as in claim 17, wherein the convex central region of the inflatable balloon is thinned relative to other portions of the balloon to cause the convex inflation geometry.

19. A stent delivery catheter as in claim 1, wherein the inflatable balloon is free from additional layers of material such as restraining or limiting members.

20. A stent delivery catheter as in claim 1, wherein the inflatable balloon includes additional layers of material such as restraining or limiting members to define the convex geometry of the central region.

21.-35. (canceled)
